(19) United States
(12) Patent Application Publication
(10) Pub. No.: US 2023/0293576 A1
(43) Pub. Date: Sep. 21, 2023

(54) ANTIVIRAL POLYMERS

(71) Applicant: The University of Manchester, Manchester (GB)

(72) Inventor: Samuel Thomas Jones, Manchester (GB)

(21) Appl. No.: 17/926,847

(22) PCT Filed: May 21, 2021
(86) PCT No.: PCT/GB2021/051247

§ 371 Date: Nov. 21, 2022

(30) Foreign Application Priority Data
May 22, 2020 (GB) 2007701.2

(51) Int. Cl. A61K 31/795; A01N 31/08; A01P 1/00; A61P 31/12; C08F 120/38; C08F 112/14

(52) U.S. Cl. CPC A61K 31/795; A01N 31/08; A01P 1/00; A61P 31/12; C08F 112/30; C08F 120/38; C08F 2438/01

(57) ABSTRACT

The present invention relates to polymer compounds that have antiviral activity. The compounds have the structural Formula I defined herein. The present invention also relates to processes for the preparation of these compounds, to compositions comprising them, and to their use in the prevention or treatment of viral infections.

### INTRODUCTION

The present invention relates to branched polymer compounds. More specifically, the present invention relates to branched, sulfonated or sulfated polymer compounds that have antiviral activity. The present invention also relates to processes for the preparation of these branched polymer compounds; to compositions comprising them; to their use in the prevention or treatment of viral infections, in particular viral infections associated with viruses which bind heparan sulfate proteoglycans (HSPG); and for disinfecting virally contaminated surfaces.

### BACKGROUND OF THE INVENTION

Viral infections are one of the leading causes of death globally, with around 20% of global deaths being caused by infectious diseases [Lozano et al., Lancet, (2012), 380(9859) 2095-2128]. Although vaccinations are effective in the prevention of viral infections, their availability may be limited and sufficient vaccine coverage can be a significant challenge in many parts of the world. After people become infected, antiviral drugs may be taken to help the immune system to fight the viral infection.

Antiviral drugs have been developed to treat viral infections caused by viruses including human immunodeficiency virus (HIV), herpes simplex virus (HSV), hepatitis B virus and influenza virus [De Clercq, Clin. Microbiol. Rev., (2016), 29(3), 695-747]. Antiviral therapies typically target viral replication mechanisms, however, due to these mechanisms being error-prone, rapid viral mutation often leads to resistance developing to antiviral therapies. Current antiviral drugs are also specific to a certain virus, or even a certain strain of a virus and so have narrow spectrum usage. Antiviral drugs, due to the targeting of intracellular mechansims shared with the host, have some intrinsic associated toxicity.

Extracellular viral mechanisms represent an alternative target to combat viral infections, with the potential for low toxicity, broad spectrum treatments. The initial stage of viral reproduction involves the attachment of a virus to a host cell. Virustatic compounds are molecules which inibit viral infection by binding reversibly to viral attachment receptors and thereby inhibiting the virus-host cell attachment. Heparin is a naturally-occurring sulfated polymer with virustatic properties. Synthetic polyanionic polymers based on heparin have also been reported [WO95/34595; WO98/03572] demonstrating broad spectrum virustatic activity. Unfortunately, the reversible nature of the viral binding typically means that virustatic drugs lack in vivo efficacy. One of the reasons is that, upon dilution in bodily fluids below the binding constant, the virustatic drug will be released from the virus and the intact virus can regain its infectivity.

Agents which are capable of the extra-cellular inhibition of viral infectivity via an irreversible mechanism are described as being virucidal. Although virucidal materials are known, many of them – such as bleach, detergents and strong acids - are toxic and cannot exert their virucidal effect without harming a host and so are limited to applications such as the sterilisation or disinfection of surfaces.

It is therefore desirable to develop biocompatible virucidal materials with broad spectrum activity and low toxicity, which are candidates for development as drugs for the prevention or treatment of viral infections.

### SUMMARY OF THE INVENTION

In one aspect, the present invention provides a branched polymer compound, as defined herein.

In another aspect, the present invention provides a composition comprising a branched polymer compound of the invention as defined herein or a salt or salts thereof.

In another aspect, the present invention provides a method of sterilisation or viral disinfection, comprising using an effective amount of a composition as defined herein.

In a further aspect, the present invention provides a device for sterilisation or viral disinfection comprising a composition as defined herein and means for dispensing the composition.

In another aspect, the present invention provides a material comprising a branched polymer as defined herein, or a salt or salts thereof.

In another aspect, the present invention provides a pharmaceutical composition comprising a branched polymer compound of the invention as defined herein, or a pharmaceutically acceptable salt or salts thereof, and one or more pharmaceutically acceptable excipients.

In another aspect, the present invention relates to a branched polymer compound of the invention as defined herein, or a pharmaceutically acceptable salt or salts thereof, or a pharmaceutical composition as defined herein, for use in the prevention or treatment of viral infections.

In another aspect, the present invention relates to a branched polymer compound of the invention as defined herein, or a pharmaceutically acceptable salt or salts thereof, or a pharmaceutical composition as defined herein, for use in the prevention or treatment of viral infections associated with one or more HSPG-binding viruses.

In another aspect, the present invention relates to the use of a branched polymer compound of the invention as defined herein, or a pharmaceutically acceptable salt or salts thereof, or a pharmaceutical composition as defined herein, in the manufacture of a medicament for use in the prevention or treatment of viral infections, in particular those associated with one or more HSPG-binding viruses.

In another aspect, the present invention relates to a method of preventing or treating viral infections, particularly those associated with one or more HSPG-binding viruses, said method comprising administering to a subject in need of such treatment a therapeutically effective amount of a branched polymer compound of the invention as defined herein, or a pharmaceutically acceptable salt or salts thereof, or a pharmaceutical composition as defined herein.

Examples of HSPG-binding viruses include herpes simplex virus (HSV), adenovirus, adeno-associated virus, human papillomavirus (HPV), respiratory syncytial virus (RSV), dengue virus, norovirus, lentivirus, human immunodeficiency virus (HIV), human cytomegalovirus (HCMV), human metapneumovirus (HMPV), human parainfluenza virus type 3 (HPIV-3), coronavirus (such as MERS-CoV, SARS-CoV, or SARS-CoV-2), foot-and-mouth disease virus, hepatitis B virus, hepatitis C virus, Ebola virus, nipah virus, Rift Valley fever virus, West Nile virus, Crimean Congo virus, Toscana virus, ZIKA virus, Chickungunya virus (CHIKV), Akabane virus (AKAV), and Schmallenberg virus (SBV).

The present invention further provides a method of synthesising a branched polymer compound, or a pharmaceutically acceptable salt or salts thereof, as defined herein.

In another aspect, the present invention provides a branched polymer compound, or a pharmaceutically acceptable salt or salts thereof, obtainable by, or obtained by, or directly obtained by a method of synthesis as defined herein.

In another aspect, the present invention provides novel intermediates as defined herein which are suitable for use in any one of the synthetic methods set out herein.

ConvenientConvenient, suitable, and optional features of any one particular aspect of the present invention are also convenientconvenient, suitable, and optional features of any other aspect.

### DETAILED DESCRIPTION OF THE INVENTION

### Definitions

Unless otherwise stated, the following terms used in the specification and claims have the following meanings set out below.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the subject to be treated.

The term “HSPG-binding virus” refers to a virus which is known to be, or is believed to be, capable of binding to heparan sulfate proteoglycans (HSPGs) on the surface of host cells after the virus has infected a host subject.

In this specification the term “alkyl” refers to aliphatic hydrocarbon groups and includes both straight and branched chain alkyl groups. References to individual alkyl groups such as “propyl” are specific for the straight chain version only and references to individual branched chain alkyl groups such as “isopropyl” are specific for the branched chain version only. For example, “C₁₋₆alkyl” includes, but is not limited to, C₁₋₄alkyl, C₁-₃alkyl, propyl, isopropyl and t-butyl.

In this specification the term “alkylene” includes both straight and branched chain divalent alkyl groups. For example, “C₁₋₃alkylene” includes, but is not limited to, methylene (—CH₂—), ethylene (—CH₂CH₂—) and propylene.

In this specification the term “alkoxy” includes both straight and branched chain alkyl groups singularly bonded to oxygen. For example, “C₁₋₄alkoxy” includes, but is not limited to, methoxy, ethoxy, isopropoxy and t-butoxy.

The term “Cₘ₋ₙ” used as a prefix, refers to any group having m to n carbon atoms.

The term “halo” refers to fluoro, chloro, bromo and iodo.

The term “haloalkyl” is used herein to refer to an alkyl group respectively in which one or more hydrogen atoms have been replaced by halogen (e.g. fluorine) atoms. Examples of haloalkyl groups include fluoroalkyl groups such as —CHF₂, —CH₂CF₃, or perfluoroalkyl/alkoxy groups such as —CF₃, or —CF₂CF₃.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In this particular embodiment, an aryl is phenyl or naphthyl, especially phenyl.

The terms “sulfonate” and “sulfate” refer to the following anionic substituent groups and encompass all salts and associated counterions thereof:

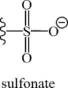

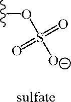

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention provides a branched polymer of Formula l:

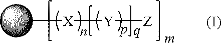

wherein

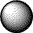

is a polyvalent core structure;
X is a monomer residue comprising at least one sulfonate or sulfate substituent;
Y is a monomer residue;
Z is a capping group;
m is greater than or equal to 3;
n is 5 to 500;
p is 1 to 500; and
q is 0 to 200; wherein if q is greater than 1, then at each occurrence, Y may be the same or different residues.

The term “polyvalent core structure” refers to any moiety having multiple branches, wherein the branches are capable of covalently bonding to the residues of the polymer chains attached thereto. In an embodiment, the polyvalent core structure has m branches. In an embodiment, the polyvalent core structure has 3 to 12 branches, such as 3 to 10, conveniently 3 to 6, most conveniently 4 or 6 branches.

In an embodiment,

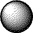

is selected from:

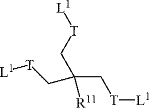

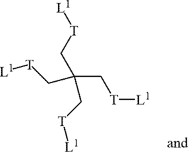

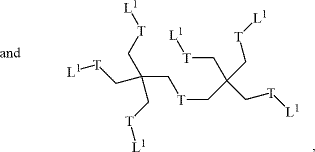

wherein
T is O, S or NH;
R¹¹ is hydrogen or C₁₋₄alkyl; and
L¹ is selected from one of the following linker groups:

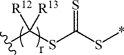

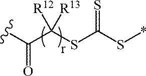

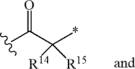

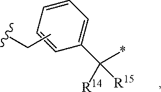

and wherein 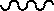
   is the point of attachment to T and * is the point of attachment to an X residue;
   R¹² and R¹³ are independently chosen from hydrogen, C₁₋₃alkyl, hydroxy, and halo;
   r is 1 to 10; and
   R¹⁴ and R¹⁵ are independently chosen from hydrogen and C₁₋₃alkyl.

In a convenient embodiment, T is O.

In a convenient embodiment, R¹¹ is hydrogen or methyl. Conveniently R¹¹ is methyl.

In an embodiment, r is 1 to 3. Conveniently, r is 2.

In an embodiment, L¹ is selected from:

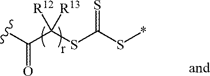

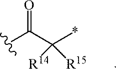

In an embodiment, L¹ is selected from:

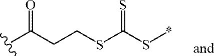

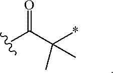

In an embodiment, L¹ is:

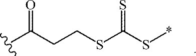

In an embodiment,

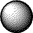

is selected from:

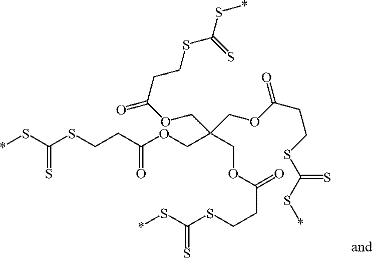

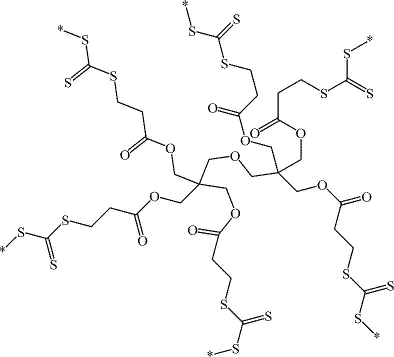 [SMILES: *SC(=S)SCCC(=O)OCC(COCC(COC(=O)CCSC(=S)S*)(COC(=O)CCSC(=S)S*)COC(=O)CCSC(=S)S*)(COC(=O)CCSC(=S)S*)COC(=O)CCSC(=S)S*]

wherein * is the point of attachment to an X residue.

The branches of the polyvalent core structure may comprise one or more additional monomer residues between the core and the first of the X residues. Therefore, in an alternative embodiment, each branch of the polyvalent core structure comprises a pendant group according to the following structure:

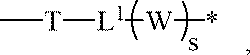

wherein T and L¹ are as described hereinbefore, W is a monomer residue, s is 0 to 200 and * is the point of attachment to an X residue. The structure of the W monomer residue is typically not limited, but may suitably be chosen from monomer residues derived from the polymerisation of monomers such as, for example, acrylates, methacrylates, acryalimdes, methacryamides, styrenes, vinyl esters, and vinyl amides. In an embodiment W is a hydrophobic monomer residue, such as derived from the polymerisation of styrene, methacrylate, or acrylamide monomers. In an embodiment, W is a monomer residue, provided that W does not encompass a residue derived from n-butyl acrylate.

In order to achieve the desired virucidal properties, it is postulated that it is necessary to have a high density of sulfonate or sulfate moieties. In the present invention this is achieved by a branched polymer according to Formula I, having 3 or more branches, with a block of sulfonated or sulphated residues attached to each branch surrounding the core structure.

As such, the branched polymers of the present invention are differentiated from multi-branched polymers, such as dendrimers. As illustrated in the schematic below (where the dashed regions represent areas of anionic (e.g. sulfonate/sulfate) substituents), a dendrimer (A) has multiple branching points related to the number of repeated branching cycles performed during its synthesis; if derivatised then this is via terminal or capping groups on the outer surface of the dendrimer, with typically, where present, only one such anionic group being present at the end of each branch. On the other hand, the branched polymers described herein have a single branching point (i.e. the polyvalent core - exemplified below (B) with a tetravalent core) from which the branches emanate, wherein each branch has from 5 to 500 (such as 50 or 100) residues, each residue bearing sulfonate/sulfate substituents, leading to the overall polymer having a high density of these anionic substituents surrounding the core.

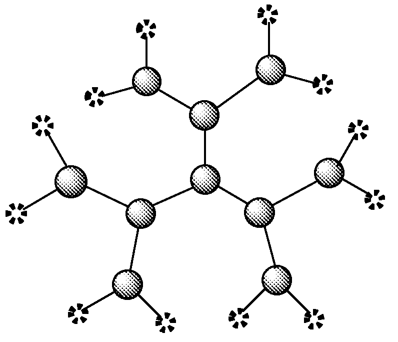

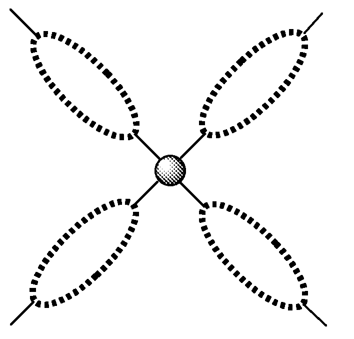

In an embodiment, the branched polymer of Formula I is not a dendrimer.

In an embodiment, X is a monomer residue comprising at least one sulfonate substituent. In an embodiment, X is a monomer residue comprising one sulfonate substituent.

Monomer residue X may be formed from the polymerisation of a C═C bond containing monomer (e.g. styrene or vinyl type monomers). Accordingly, in an embodiment, X has a structure according to Formula II:

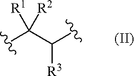

wherein
R¹ is aryl, C₁₋₂₀alkyl, C₁₋₂₀alkylene-aryl, C(O)OR⁴, C(O)NHR⁴, OC(O)R⁴, NHC(O)R⁴ or sulfonate;
R² and R³ are independently selected from hydrogen and C₁₋₄alkyl; and
R⁴ is C₁₋₁₀alkyl or aryl; and wherein
each of the aryl, C₁₋₂₀alkyl, C₁₋₂₀alkylene-aryl or R⁴ groups is substituted with one or more sulfonate or sulfate groups, and is optionally substituted with one or more substituents selected from hydroxy, halo, C₁₋₄alkyl, C₁₋₄alkoxy, aryl, and cyano.

In an embodiment, R¹ is aryl, C₁₋₂₀alkyl, C₁₋₂₀alkylene-aryl, C(O)OR⁴, C(O)NHR⁴, OC(O)R⁴, NHC(O)R⁴, or sulfonate; R² and R³ are independently selected from hydrogen and C₁₋₄alkyl; and R⁴ is C₁₋₁₀alkyl or aryl; and wherein each of the aryl, C₁₋₂₀alkyl, C₁-₂₀alkylene-aryl, or R⁴ groups is substituted with one or more sulfonate groups (conveniently one sulfonate group), and is optionally substituted with one or more substituents selected from hydroxy, halo, C₁₋₄alkyl, C₁₋₄alkoxy, aryl, and cyano.

In an embodiment, R¹ is phenyl, C₁₋₂₀alkyl, C₁₋₂₀alkylene-phenyl, C(O)OR⁴, or C(O)NHR⁴, wherein R⁴ is C₁₋₁₀alkyl or phenyl and wherein each of the phenyl, C₁₋₂₀alkyl, C₁₋₂₀alkylene-phenyl, or R⁴ groups is substituted with one or more sulfonate groups and is optionally substituted with one or more substituents selected from hydroxy, halo, C₁-₄alkyl, C₁₋₄alkoxy, aryl, and cyano.

In an embodiment, R¹ is phenyl, C(O)OR⁴ or C(O)NHR⁴, wherein R⁴ is C₁₋₁₀alkyl and each of the phenyl or R⁴ groups is substituted with one or more sulfonate groups and is optionally substituted with one or more substituents selected from hydroxy, halo, C₁₋₄alkyl, C₁₋₄alkoxy, and cyano.

In an embodiment, R¹ is phenyl substituted with one sulfonate group and optionally substituted with one or more substituents selected from hydroxy, halo, C₁-₄alkyl, C₁₋₄alkoxy, and cyano. In an embodiment, R¹ is phenyl substituted with one sulfonate group.

In an embodiment, R¹ is C(O)OR⁴ wherein R⁴ is C₁₋₁₀alkyl substituted with one sulfonate group and optionally substituted with one or more substituents selected from hydroxy, halo, C₁₋₄alkoxy, and cyano.

In an embodiment, R¹ is C(O)NHR⁴, wherein R⁴ is C₁₋₁₀alkyl substituted with one sulfonate group and optionally substituted with one or more substituents selected from hydroxy, halo, C₁₋₄alkoxy, and cyano.

In an embodiment, R¹ is selected from:

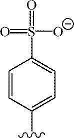

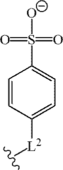

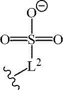

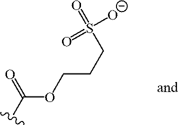

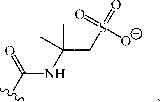

wherein L² is C₁₋₂₀alkylene.

In an embodiment, R¹ is selected from:

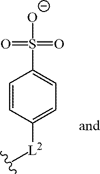

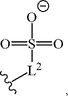

wherein L² is C₁₋₂₀alkylene.

In an embodiment, R² and R³ are independently selected from hydrogen and methyl. In an embodiment, R² is hydrogen. In an embodiment, R² is methyl. In an embodiment, R³ is hydrogen. In an embodiment, R² is hydrogen and R³ is hydrogen. In an embodiment, R² is methyl and R³ is hydrogen.

In an embodiment, X is selected from:

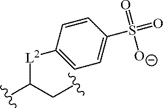

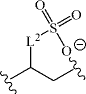

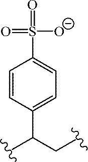

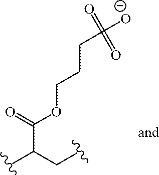

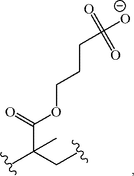

wherein L² is C₁₋₂₀alkylene.

In a convenient embodiment, X is selected from:

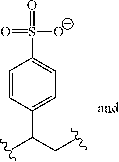

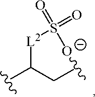

wherein L² is C₁₋₂₀alkylene.

In a further convenient embodiment, X is:

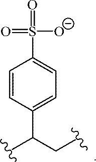

In an embodiment, n is 5 to 400, such as 5 to 300, 10 to 250, or conveniently 20 to 150. In a convenient embodiment n is 10 to 100, such as 10, 20, 30, 40, 50, 60, 70, 80, 90 or 100. In a more convenient embodiment, n is 30 to 100, such as 50 to 100. In a convenient embodiment, n is 30. In a convenient embodiment, n is 50. In a convenient embodiment, n is 100.

Additional monomer residues (Y) may also be present on each branch of the branched polymer according to Formula I. Whilst not essential for the virucidal activity of the branched polymers of the present invention, the Y monomer residues can be incorporated into the structure of the branched polymer according to Formula I to potentially modify other properties of the branched polymer, as may be desirable. Therefore, the structure of the Y monomer residue is not limited, but may suitably be chosen from monomer residues derived from the polymerisation of monomers such as, for example, acrylates, methacrylates, acryalimdes, methacryamides, styrenes, vinyl esters, vinyl amides and ethylene glycols.

In an embodiment, p is 5 to 400, such as 5 to 300, 10 to 250, or conveniently 20 to 150.

Each branch of the branched polymer according to Formula I comprises a block of X residues, optionally followed by a block of Y residues. q is the number of Y residues; q is 0 to 200; wherein if q is greater than 1, then at each occurrence, Y may be the same or different residues. When no Y residues are present, then q = 0. When a single block of Y residues (Y1) are present, then q = 1. In one embodiment, q is 0. In another embodiment, q is 1. In an embodiment, q is 1 to 200, such as 1 to 100 or 1 to 50. In an embodiment, q is 2 to 200, such as 2 to 100 or 2 to 50; wherein at each occurrence Y may be the same or different residues.

When q is greater than 1, then the second block of Y residues (Y2) and further blocks of Y residues (Y3, Y4, Y5 etc) may be the same or different residues to Y1 and also to each other. Therefore, in an embodiment, q is 0 to 200, such as 0 to 100 or 0 to 50, wherein if q is greater than 1, then at each occurrence, Y may be the same or different residues. For the avoidance of doubt, the present invention covers both block co-polymers (as exemplified in (A) below by blocks of three different residues (Y1, Y2 and Y3), where each p is independently chosen from 2 to 500), and mixed polymers (as exemplified in (B) below by an alternating mixture of two different residues (Y1 and Y2)):

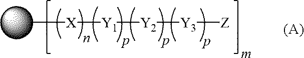

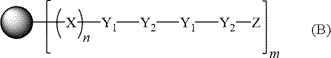

The group Z is a capping group. The term “capping group” generally refers to any group which is present at the end of the polymer branch, distal from the core. The capping group may be a product of the polymerisation used to synthesise the polymer branches. In an embodiment, Z is selected from hydrogen, hydroxyl, bromo, chloro, or one of the following groups:

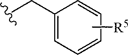

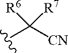

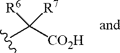

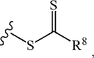

wherein:
R⁵ is hydrogen, halo, cyano, CO₂H, C₁₋₃haloalkyl, C₁₋₃alkylene-OH, or C₁₋₃alkylene-NH₂;
R⁶ and R⁷ are independently chosen from hydrogen and C₁₋₃alkyl optionally substituted with cyano, halo, or CO₂H;
R⁸ is S-C₁₋₁₅alkyl, S-aryl, NR⁹R¹⁰ or aryl, said C₁₋₁₅alkyl and aryl groups being optionally substituted with one or more substituents selected from hydroxy, C₁₋₃alkyl, halo, cyano,
CO₂H, and C₁₋₃haloalkyl;
R⁹ is hydrogen or C₁₋₃alkyl; and
R¹⁰ is hydrogen, C₁₋₁₂alkyl or aryl, said C₁₋₁₂alkyl and aryl groups being optionally substituted with one or more substituents selected from hydroxy, C₁₋₃alkyl, halo, cyano, CO₂H, and C₁₋₃haloalkyl.

In an embodiment, R⁵ is cyano, CO₂H, C₁₋₃haloalkyl, C₁₋₃alkylene-OH or C₁-₃alkylene-NH₂, such as C₁₋₃alkylene-OH (conveniently CH₂—OH).

In an embodiment, R⁶ and R⁷ are independently chosen from hydrogen and methyl. In an embodiment, R⁶ and R⁷ are both hydrogen. In an embodiment, R⁶ and R⁷ are both methyl.

In an embodiment, R⁸ is S-C₁₋₆alkyl, S-phenyl, NR⁹R¹⁰ or phenyl, said C₁₋₆alkyl and phenyl groups being optionally substituted with one or more substituents selected from hydroxy, C₁₋₃alkyl, halo, cyano, CO₂H, and C₁₋₃haloalkyl.

In an embodiment, Z is bromo or chloro or is selected from one of the following groups:

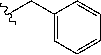

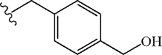

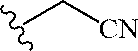

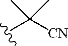

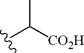

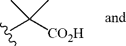

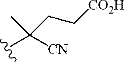

As described hereinafter, the branched polymers according to Formula I may typically be prepared by living free-radical polymerisation. Two suitable approaches are reversible addition fragmentation chain transfer (RAFT) polymerisation and atom transfer radical polymerisation (ATRP).

RAFT polymerisation uses thiocarbonylthio chain transfer agents (CTAs) of general Formula R—(C═S)—SR′ to control the free radical polymerisation. In the case of the branched polymers of the present invention, either R or R′ may form part of the core structure. Therefore, in an embodiment, L¹ is selected from:

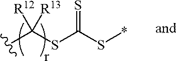

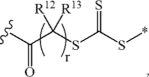

wherein R¹², R¹³ and r are as defined hereinbefore and Z is selected from:

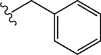

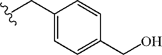

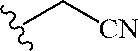

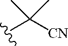

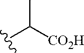

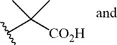

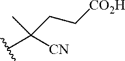

In an alternative embodiment, L¹ is

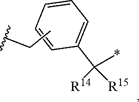

and Z is:

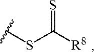

wherein R⁸, R¹⁴ and R¹⁵ are as defined hereinbefore.

In an embodiment, there is provided a branched polymer of Formula IA:

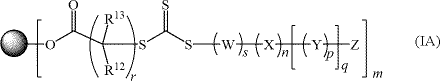

wherein

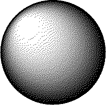

is a polyvalent core structure;
W is a monomer residue;
X is a monomer residue comprising at least one sulfonate or sulfate substituent;
Y is a monomer residue;
Z is selected from one of the following groups:

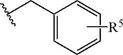

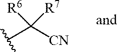

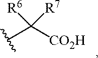

wherein:
   R⁵ is hydrogen, halo, cyano, CO₂H, C₁₋₃haloalkyl, C₁₋₃alkylene-OH or C₁-₃alkylene-NH₂; and
   R⁶ and R⁷ are independently chosen from hydrogen and C₁₋₃alkyl optionally substituted with cyano, halo, or CO₂H;
R¹² and R¹³ are independently chosen from hydrogen, C₁₋₃alkyl, hydroxy, and halo;
r is 1 to 10;
m is greater than or equal to 3;
s is 0 to 100;
n is 5 to 500;
p is 1 to 500; and
q is 0 to 200; wherein if q is greater than 1, then at each occurrence, Y may be the same or different residues.

For the branched polymers of Formula IA, W, X, Y, Z, R¹², R¹³, r, m, s, n, p and q may be as described herein hereinbefore or hereinafter.

In a convenient embodiment, there is provided a branched polymer of Formula IA, wherein:
R⁵ is hydrogen or C₁₋₃alkylene-OH;
R⁶ and R⁷ are independently chosen from hydrogen and C₁₋₃alkyl;
R¹² and R¹³ are independently chosen from hydrogen and C₁₋₃alkyl;
r is 1 to 3 (such as 2); and
W, X, Y, m, s, n, p and q are as described herein hereinbefore or hereinafter.

In an embodiment, there is provided a branched polymer of Formula IB:

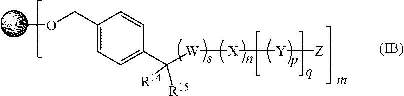

wherein

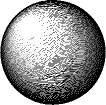

is a polyvalent core structure;
W is a monomer residue;
X is a monomer residue comprising at least one sulfonate or sulfate substituent;
Y is a monomer residue;
Z is:

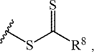

wherein R³ is S-C₁₋₁₅alkyl, S-aryl, NR⁹R¹⁰ or aryl, said C₁₋₁₅alkyl and aryl groups being optionally substituted with one or more substituents selected from hydroxy, C₁₋₃alkyl, halo, cyano, CO₂H, and C₁₋₃haloalkyl;
R⁹ is hydrogen or C₁₋₃alkyl; and
R¹⁰ is hydrogen, C₁₋₁₂alkyl or aryl, said C₁₋₁₂alkyl and aryl groups being optionally substituted with one or more substituents selected from hydroxy, C₁₋₃alkyl, halo, cyano, CO₂H, and C₁₋₃haloalkyl;
R¹⁴ and R¹⁵ are independently chosen from hydrogen and C₁₋₃alkyl;
m is greater than or equal to 3;
s is 0 to 100;
n is 5 to 500;
p is 1 to 500; and
q is 0 to 200; wherein if q is greater than 1, then at each occurrence, Y may be the same or different residues.

For the branched polymers of Formula IB, W, X, Y, Z, R¹⁴, R¹⁵, m, s, n, p and q may be as described herein hereinbefore or hereinafter.

ATRP typically uses alkyl halides to initiate the free radical chain polymerisation. Therefore, in an embodiment, L¹ is:

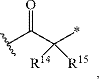

wherein R¹⁴ and R¹⁵ are as defined hereinbefore, and Z is bromo or chloro.

In an embodiment, there is provided a branched polymer of Formula IC:

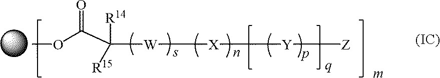

wherein

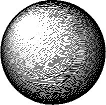

is a polyvalent core structure;
W is a monomer residue;
X is a monomer residue comprising at least one sulfonate or sulfate substituent;
Y is a monomer residue;
Z is bromo or chloro;
R¹⁴ and R¹⁵ are independently chosen from hydrogen and C₁₋₃alkyl;
m is greater than or equal to 3;
s is 0 to 100;
n is 5 to 500;
p is 1 to 500; and
q is 0 to 200; wherein if q is greater than 1, then at each occurrence, Y may be the same or different residues;
provided that W does not encompass a residue derived from n-butyl acrylate.

For the branched polymers of Formula lC, W, X, Y, Z, R¹⁴, R¹⁵, m, s, n, p and q may be as described hereinbefore or hereinafter.

Alternatively, Z may be a capping group not resulting from living free-radical polymerisation. After the X residues, and optionally Y residues, have been assembled, then other chemistry or chemistries may be carried out to introduce alternative capping groups. These other chemistries may be different polymerisation reactions or other chemical reactions or transformations as may be apparent to one skilled in the art. For instance, and purely for exemplary purposes, if the end group of each polymer branch is a bromo, then the bromo group may be substituted by a nucleophilic substitution reaction to introduce an alternative capping group. The scope of such alternative capping groups is not limited and a wide variety of capping groups could be introduced dependent on the desired properties of the resultant branched polymer compound. For example, Z may be a dye molecule or a radiolabelled moiety.

The branched polymer according to Formula I has at least three branches bearing sulfonated or sulphated X residues. Therefore, m is greater than or equal to 3. In an embodiment, m is 3 to 16, such as 3 to 12, or 4 to 10. In an embodiment, m is 3, 4, 5, 6, 7, 8, 9, 10, 11, 12, 13, 14, 15, or 16; conveniently m is 4, or m is 6. Conveniently, m is 4. Conveniently, m is 6.

In a further embodiment, there is provided a branched polymer according to Formula I, wherein:

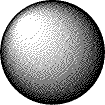

is:

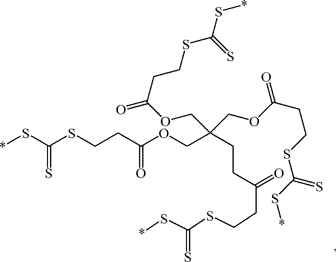

wherein * is the point of attachment to an X residue;
X is selected from:

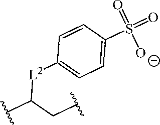

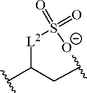

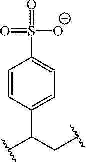

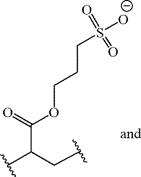

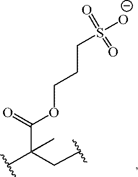

wherein L² is C₁₋₂₀alkylene;
Z is:

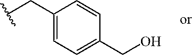

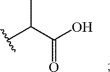

and m is 4.

In a further embodiment, there is provided a branched polymer according to Formula I, wherein:

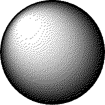

is:

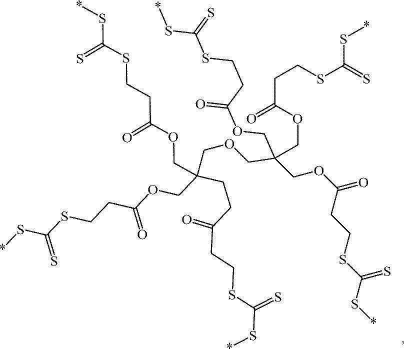

wherein * is the point of attachment to an X residue;
X is selected from:

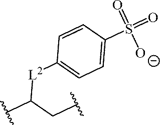

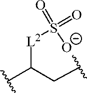

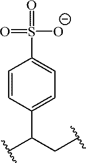

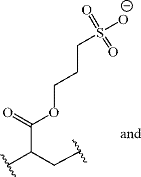

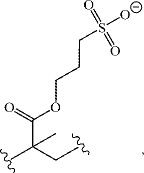

wherein L² is C₁₋₂₀alkylene;
Z is:

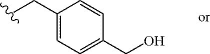

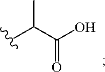

and m is 6.

In a further embodiment, there is provided a branched polymer according to Formula I, wherein:

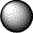

is selected from:

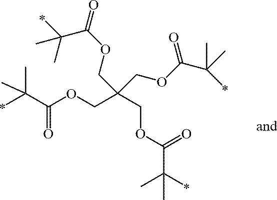

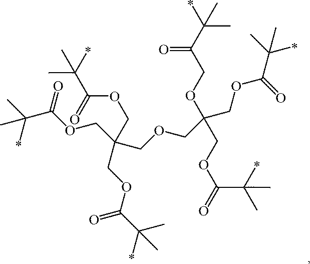

wherein * is the point of attachment to an X residue;
X is selected from:

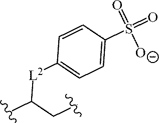

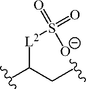

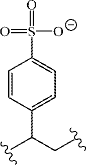

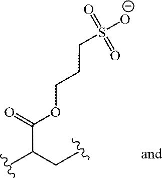

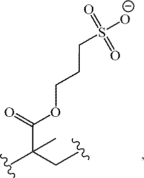

wherein L² is C₁₋₂₀alkylene;
Z is bromo; and
m is 4 or 6.

In a further embodiment, there is provided a branched polymer according to Formula I, wherein X is:

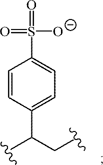

n is 10 to 100 (such as 10, 30, 50 or 100), q is 0 and m is 4 or 6 (such as 4).

In a further embodiment, there is provided a branched polymer according to Formula I, wherein X is:

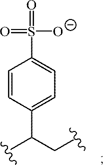

n is 30, q is 0 and m is 4.

In another embodiment, there is provided a branched polymer according to Formula I, wherein X is:

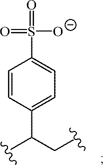

n is 50, q is 0 and m is 4.

In a yet further embodiment, there is provided a branched polymer according to Formula I, wherein X is:

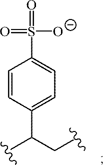

n is 100, q is 0 and m is 4.

Particular compounds of the present invention include any one of the example branched polymer compounds listed herein.

Suitable or convenient features of any compounds of the present invention may also be suitable features of any other aspect.

The branched polymer compounds according to Formula I comprise multiple sulfate or sulfonate substituents. These substituents may be present as their acid or salt forms, or a mixture thereof, and all such branched compounds are included within the scope of the present invention. Therefore, in an embodiment, the present invention provides a compound of Formula I as described herein, or a salt or salts thereof. In a further embodiment, the present invention provides a compound of Formula I as described herein, or a pharmaceutically acceptable salt or salts thereof. Where the sulfonate or sulfate substituents are present in their salt form, then due to the presence of multiple sulfonate/sulfate substituents, the salts thereof may be the same or different.

A suitable salt of a compound of the invention is a metal salt, such as an alkali metal salt, or a salt with an organic base. A suitable pharmaceutically acceptable salt of a compound of the invention is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular Formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (-)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z- isomers). It is to be understood that the present invention encompasses all optical, diastereoisomers and geometric isomers and mixtures thereof that possess virucidal activity.

The present invention also encompasses compounds of the invention as defined herein which comprise one or more isotopic substitutions. For example, H may be in any isotopic form, including ¹H, ²H (D) and ³H (T); C may be in any isotopic form including ¹²C, ¹³C, and ¹⁴C; and O may be in any isotopic form, including ¹⁶O and ¹⁸O; and the like.

It is also to be understood that certain compounds of the invention may exist in solvated as well as unsolvated forms such as, for example, hydrated forms. It is to be understood that the invention encompasses all such solvated forms that possess virucidal activity.

It is also to be understood that branched polymers of the invention may be amorphous, partially crystalline or crystalline, depending, for example, on the tacticity of the polymer branches. Crystalline compounds of the invention may exhibit polymorphism, and the invention encompasses all such forms that possess virucidal activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol, imine/enamine, amide/imino alcohol, amidine/amidine, nitroso/oxime, thioketone/enethiol, and nitro/aci-nitro.

### Synthesis

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃.OEt₂. A suitable alternative protecting group for a primary amino group is, for example, a phthaloyl group which may be removed by treatment with an alkylamine, for example dimethylaminopropylamine, or with hydrazine.

The person skilled in the art will recognise that the compounds of the invention may be prepared, in known manner, in a variety of ways. Compounds of Formula I can be prepared by the methods given below, by the methods given in the experimental or by analogous methods. The routes described are merely illustrative of some of the methods that can be employed for the synthesis of compounds of Formulae I and the person skilled in the art will appreciate that the order of the reaction steps is not limited to those described.

The branched polymers according to Formula I can be formed by any suitable standard polymerisation process as may be determined by the person skilled in the art. Suitable polymerisation processes include living polymerisation techniques, due to the control offered in preparing polymer branches with low polydispersity and the ability to synthesise block-copolymer branches. Living free-radical polymerisation and in particular RAFT and ATRP are convenient techniques. With RAFT and ATRP, the polymerisation technique employed will influence the core structure on which the branched polymer is synthesised.

RAFT polymerisation uses thiocarbonylthio chain transfer agents of general Formula R—(C═S)—SR′ to control the free radical polymerisation. In the case of the branched polymers of the present invention, either R or R′ may form part of the core structure. Therefore, branched polymers according to Formula I may be synthesised by RAFT polymerisation according to the schematic shown below, whereby _{〰} represents the end of the polymer branch attached to the core:

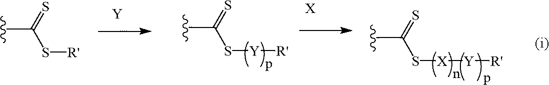

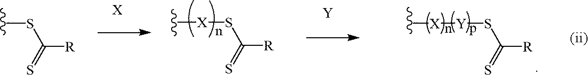

For RAFT polymerisation according to the synthetic approach (i) above, a branch of the core will have the structure:

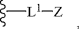

wherein L¹ may typically be selected from:

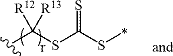

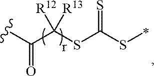

wherein R¹², R¹³ and r are as defined hereinbefore,

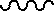

represents the point of attachment to the core and * is the point of attachment to Z, and Z may typically be selected from:

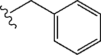

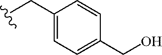

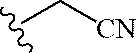

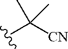

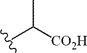

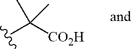

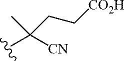

For RAFT polymerisation according to the synthetic approach (ii) above, a branch of the core will have the structure:

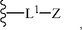

wherein L¹ may typically be:

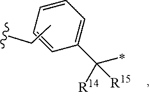

wherein

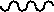

represents the point of attachment to the core and * is the point of attachment to Z and Z may typically be:

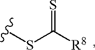

wherein R⁸, R¹⁴ and R¹⁵ are as defined hereinbefore.

ATRP typically uses alkyl halides to initiate the free radical chain polymerisation. Therefore, branched polymers according to Formula I may be synthesised by ATRP according to the schematic shown below, whereby

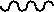

represents the end of the polymer branch attached to the core:

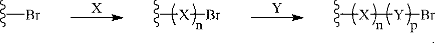

For such ATRP polymer synthesis, a branch of the core will have the structure:

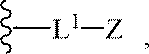

wherein for the synthesis approach above, L¹ may typically be:

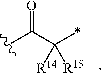

wherein R¹⁴ and R¹⁵ are as defined hereinbefore,

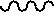

represents the point of attachment to the core and * is the point of attachment to Z and Z may be halo such as bromo.

Suitable reaction conditions for carrying out RAFT and ATRP syntheses will be readily determined by a person skilled in the art and by reference to the Examples provided below.

### Compositions

The branched polymers of the present invention have virucidal properties which make them potentially useful for a number of applications. Pharmaceutical compositions comprising a branched polymer according to Formula I have applications in the prevention and treatment of viral infections in humans and other animals; such pharmaceutical compositions are described separately below.

Other applications include the sterilisation and disinfection of surfaces contaminated, or suspected to be contaminated, by viruses.

In an aspect of the invention, there is provided a composition comprising a branched polymer as defined herein, or a salt or salts thereof. In an embodiment, there is provided a composition comprising an effective amount of a branched polymer as defined herein, or a salt or salts thereof, and at least one carrier. An “effective amount” refers to an amount sufficient for destroying viruses. Suitably, a carrier may be a stabiliser, wetting agent, emulsifier, thickener, fragrance, colourant or mixtures thereof.

In an embodiment, the composition is Formulated as a solution, a gel, a foam, or an emulsion. In an embodiment, the composition is a sterilising solution or a disinfecting solution.

### Pharmaceutical Compositions

The compounds of the invention may be Formulated into pharmaceutical compositions prior to administration to a patient. Therefore, according to a further aspect of the invention there is provided a pharmaceutical composition which comprises a branched polymer as defined herein, or a pharmaceutically acceptable salt or salts thereof, and one or more pharmaceutically acceptable excipients.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The compositions of the invention may be in a form suitable for oral use (for example as tablets, capsules, caplets, pills, troches, powders, syrups, elixirs, suspensions, solutions, emulsions, sachets, and cachets), for topical use (for example as creams, ointments, lotions, solutions, pastes, sprays, foams, and gels), for transdermal administration such as via transdermal patches, for administration by inhalation (for example as a dry powders, aerosols, suspensions, and solutions), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

As used herein, “pharmaceutically acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the Formulation and what other ingredients are present in the Formulation may be chosen for their ability to enhance patient compliance.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington’s Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington’s Pharmaceutical Sciences (Mack Publishing Company).

In one embodiment, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In another embodiment, the invention is directed to a dosage form adapted for administration to a patient parenterally including subcutaneous, intramuscular, intravenous, intrathecal or intradermal. Pharmaceutical Formulations adapted for parenteral administration include aqueous and non-aqueous sterile injection solutions which may contain anti-oxidants, buffers, bacteriostats, and solutes that render the Formulation isotonic with the blood of the intended recipient; and aqueous and non-aqueous sterile suspensions which may include suspending agents and thickening agents. The Formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In another embodiment, the invention is directed to a dosage form adapted for topical administration to a patient. For example, the compound of the invention may be applied topically as a cream, an ointment, a lotion, a solution, a paste, a spray, a foam, or a gel. Suitable topical Formulations may typically comprise one or more of emollients, solubilising agents, humectants, gelling agents, preservatives, permeation enhancers, chelating agents, antioxidants, buffering agents and solvents.

In another embodiment, the invention is directed to a dosage form adapted for administration to a patient by inhalation (e.g. nasal or pulmonary). For example, the compound of the invention may be inhaled into the lungs as a dry powder, an aerosol, a suspension, or a solution. For administration by inhalation, the pharmaceutical compositions can be conveniently delivered in the form of an aerosol spray, from pressurized packs or via a nebulizer, or with the use of suitable propellant, e.g. dichlorodifluoromethane, trichlorofluoromethane, dichlorotetrafluoroethane, carbon dioxide, propane, butane or other suitable gases or mixture of gases. The particle size of the aerosol is not critical, but particles under about 3-5 microns in diameter are convenient for deep penetration into the pulmonary system. Continuous administation in a hospital environment or periodic administration, e.g. at 1 to 6 hour intervals, will typically allow the antiviral polymer compounds as described herein to act effectively in the subject’s pulmonary system.

An effective amount of a compound of the present invention for use in therapy of proliferative disease is an amount sufficient to symptomatically relieve in a warm-blooded animal, particularly a human, the symptoms of infection, to slow the progression of infection, or to reduce in patients with symptoms of infection the risk of getting worse.

The amount of active ingredient that is combined with one or more excipients to produce a single dosage form will necessarily vary depending upon the host treated and the particular route of administration. For example, a Formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the Formula I will naturally vary according to the viral infection being treated, the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The compounds of the invention have been found to have antiviral activity. As a consequence, they are potentially useful in the treatment of viral infections.

Thus, in one aspect, the present invention provides a branched polymer as defined herein, or a pharmaceutically acceptable salt or salts thereof, or a pharmaceutical composition as defined herein, for use in the prevention or treatment of viral infections.

Heparan sulfate proteoglycans (HSPGs) are proteoglycan receptors located on the surface of almost all eukaryotic cells. A large number of extrcellular proteins bind to cell-surface HSPGs. A number of viruses also utilise HSPGs as attachment factors. These viruses typically have capsid proteins capable of binding to HSPGs on the surface of host cells, which leads to viral entry of the host cell. It is postulated that the branched polymers of the present invention are able to mimic the HSPG-viral interaction and that they preferentially bind to and destroy HSPG-binding viruses. Therefore, in another aspect, the present invention provides a branched polymer as defined herein, or a pharmaceutically acceptable salt or salts thereof, or a pharmaceutical composition as defined herein, for use in the prevention or treatment of viral infections associated with one or more HSPG-binding viruses.

HSPG-binding viruses include, but are not limited to, herpes simplex virus (HSV), adenovirus, adeno-associated virus, human papillomavirus (HPV), respiratory syncytial virus (RSV), dengue virus, norovirus, lentivirus, human immunodeficiency virus (HIV), human cytomegalovirus (HCMV), human metapneumovirus (HMPV), human parainfluenza virus type 3 (HPIV-3), coronavirus (such as MERS-CoV, SARS-CoV, or SARS-CoV-2), foot-and-mouth disease virus, hepatitis B virus, hepatitis C virus, Ebola virus, nipah virus, Rift Valley fever virus, West Nile virus, Crimean Congo virus, Toscana virus, ZIKA virus, Chickungunya virus (CHIKV), Akabane virus (AKAV), and Schmallenberg virus (SBV).

Coronaviruses include alpha, beta, gamma, and delta coronaviruses. In particular, the present invention is directed to human alpha and beta coronaviruses such as the human alpha coronaviruses referred to as HCoV-229E and HCoV-NL63 and the human beta coronaviruses referred to as HCoV-OC43, HCoV-HKU1, MERS-CoV, SARS-CoV and SARS-CoV-2. SARS-CoV-2 is severe acute respiratory syndrome coronavirus 2 which was originally identified in China at the end of 2019; it has also been referred to as 2019-nCoV and the disease it causes is referred to as COVID-19.

The branched polymers of the present invention may potentially be able to exert antiviral activity against viruses which are not dependent on HSPG-binding to effect viral entry into the host cells. Recent studies have shown that sulfonated nanomaterials may have antiviral activity beyond HSPG-dependent viruses, for example against influenza virus or vesicular stomatitis virus [Cagno et al., Antimicrobial Agents and Chemotherapy, (2020) DOI: 10.1128/AAC.02001-20].

Therefore, in an embodiment, there is provided a branched polymer as defined herein, or a pharmaceutically acceptable salt or salts thereof, or a pharmaceutical composition as defined herein, for use in the prevention or treatment of viral infections associated with influenza (such as Influenza A H3N2 or H1N1 virus), adeno-associated virus (AAV), Newcastle disease virus (NDV), and vesicular stomatitis virus (VSV).

Throughout this specification, where mention is made of a human virus, the scope of the invention covers the same virus in non-human animal species. For example, reference to human immunodeficiency virus is intended to cover immunodeficiency viruses in other animals such as feline immunodeficiency virus or bovine immunodeficiency virus.

In one aspect, the present invention provides a branched polymer as defined herein, or a pharmaceutically acceptable salt or salts thereof, or a pharmaceutical composition as defined herein, for use in the prevention or treatment of viral infections associated with one or more viruses selected from herpes simplex virus (HSV), adenovirus, adeno-associated virus, human papillomavirus (HPV), respiratory syncytial virus (RSV), dengue virus, norovirus, lentivirus, human immunodeficiency virus (HIV), human cytomegalovirus (HCMV), human metapneumovirus (HMPV), human parainfluenza virus type 3 (HPIV-3), coronavirus (such as MERS-CoV, SARS-CoV, or SARS-CoV-2), foot-and-mouth disease virus, hepatitis B virus, hepatitis C virus, Ebola virus, nipah virus, Rift Valley fever virus, West Nile virus, Crimean Congo virus, Toscana virus, ZIKA virus, Chickungunya virus (CHIKV), Akabane virus (AKAV), Schmallenberg virus (SBV), influenza (such as Influenza A H3N2 or H1N1 virus), adeno-associated virus (AAV), Newcastle disease virus (NDV), and vesicular stomatitis virus (VSV).

In a convenient embodiment, the present invention provides a branched polymer as defined herein, or a pharmaceutically acceptable salt or salts thereof, or a pharmaceutical composition as defined herein, for use in the prevention or treatment of viral infections associated with one or more viruses selected from herpes simplex virus (HSV), respiratory syncytial virus (RSV), human cytomegalovirus (HCMV), hepatitis C virus, dengue virus, ZIKA virus, Chickungunya virus (CHIKV), or coronavirus (such as SARS-CoV-2). In a convenient embodiment, the present invention provides a branched polymer as defined herein, or a pharmaceutically acceptable salt or salts thereof, or a pharmaceutical composition as defined herein, for use in the prevention or treatment of viral infections associated with one or more viruses selected from HSV-1, HSV-2, RSV, HCMV, or SARS-CoV-2. In a further convenient embodiment, the present invention provides a branched polymer as defined herein, or a pharmaceutically acceptable salt or salts thereof, or a pharmaceutical composition as defined herein, for use in the prevention or treatment of viral infections associated with SARS-CoV-2.

In another aspect, the present invention relates to the use of a branched polymer as defined herein, or a pharmaceutically acceptable salt or salts thereof, or a pharmaceutical composition as defined herein, in the manufacture of a medicament for use in the prevention or treatment of viral infections, for example, herpes simplex virus (HSV), adenovirus, adeno-associated virus, human papillomavirus (HPV), respiratory syncytial virus (RSV), dengue virus, norovirus, lentivirus, human immunodeficiency virus (HIV), human cytomegalovirus (HCMV), human metapneumovirus (HMPV), human parainfluenza virus type 3 (HPIV-3), coronavirus (such as MERS-CoV, SARS-CoV, or SARS-CoV-2), foot-and-mouth disease virus, hepatitis B virus, hepatitis C virus, Ebola virus, nipah virus, Rift Valley fever virus, West Nile virus, Crimean Congo virus, Toscana virus, ZIKA virus, Chickungunya virus (CHIKV), Akabane virus (AKAV), Schmallenberg virus (SBV), influenza (such as Influenza A H3N2 or H1N1 virus), adeno-associated virus (AAV), Newcastle disease virus (NDV), and vesicular stomatitis virus (VSV).

In a convenient embodiment, the present invention relates to the use of a branched polymer as defined herein, or a pharmaceutically acceptable salt or salts thereof, or a pharmaceutical composition as defined herein, in the manufacture of a medicament for use in the prevention or treatment of viral infections associated with one or more viruses selected from herpes simplex virus (HSV), respiratory syncytial virus (RSV), human cytomegalovirus (HCMV), or coronavirus (such as SARS-CoV-2). In a further convenient embodiment, the present invention relates to the use of a branched polymer as defined herein, or a pharmaceutically acceptable salt or salts thereof, or a pharmaceutical composition as defined herein, in the manufacture of a medicament for use in the prevention or treatment of viral infections associated with SARS-CoV-2.

In another aspect, the present invention relates to a method of preventing or treating a viral infection, such as a viral infection associated with herpes simplex virus (HSV), adenovirus, adeno-associated virus, human papillomavirus (HPV), respiratory syncytial virus (RSV), dengue virus, norovirus, lentivirus, human immunodeficiency virus (HIV), human cytomegalovirus (HCMV), human metapneumovirus (HMPV), human parainfluenza virus type 3 (HPIV-3), coronavirus (such as MERS-CoV, SARS-CoV, or SARS-CoV-2), foot-and-mouth disease virus, hepatitis B virus, hepatitis C virus, Ebola virus, nipah virus, Rift Valley fever virus, West Nile virus, Crimean Congo virus, Toscana virus, ZIKA virus, Chickungunya virus (CHIKV), Akabane virus (AKAV), Schmallenberg virus (SBV), influenza (such as Influenza A H3N2 or H1N1 virus), adeno-associated virus (AAV), Newcastle disease virus (NDV), and vesicular stomatitis virus (VSV), wherein said method comprises administering to a subject in need of such treatment a therapeutically effective amount of a branched polymer as defined herein, or a pharmaceutically acceptable salt or salts thereof, or a pharmaceutical composition as defined herein.

In a convenient embodiment, the present invention relates to a method of preventing or treating a viral infection associated with one or more viruses selected from herpes simplex virus (HSV), respiratory syncytial virus (RSV), human cytomegalovirus (HCMV), and coronavirus (such as SARS-CoV-2), wherein said method comprises administering to a subject in need of such treatment a therapeutically effective amount of a branched polymer as defined herein, or a pharmaceutically acceptable salt or salts thereof, or a pharmaceutical composition as defined herein. In a further convenient embodiment, the present invention relates to a method of preventing or treating a viral infection associated with SARS-CoV-2, wherein said method comprises administering to a subject in need of such treatment a therapeutically effective amount of a branched polymer as defined herein, or a pharmaceutically acceptable salt or salts thereof, or a pharmaceutical composition as defined herein.

In one embodiment, the subject in need of treatment with a branched polymer or pharmaceutical composition as described herein, is a human. In another embodiment, the subject is an animal, such as a mammal, in need of such treatment.

### Non-Therapeutic Uses

In another aspect, the present invention provides a method of sterilisation or viral disinfection, comprising using an effective amount of a branched polymer or a composition comprising a branched polymer, as described herein. In an embodiment, the method of sterilisation or viral disinfection comprises the steps of (i) providing at least one branched polymer according to Formula I, or a salt or salts thereof, or a composition comprising a branched polymer according to Formula I, or a salt or salts thereof, (ii) contacting a surface contaminated by a virus, or suspected of being contaminated by a virus, with the branched polymer or composition provided in step (i). Conveniently, the virus is herpes simplex virus (HSV), adenovirus, adeno-associated virus, human papillomavirus (HPV), respiratory syncytial virus (RSV), dengue virus, norovirus, lentivirus, human immunodeficiency virus (HIV), human cytomegalovirus (HCMV), human metapneumovirus (HMPV), human parainfluenza virus type 3 (HPIV-3), coronavirus (such as MERS-CoV, SARS-CoV, or SARS-CoV-2), foot-and-mouth disease virus, hepatitis B virus, hepatitis C virus, Ebola virus, nipah virus, Rift Valley fever virus, West Nile virus, Crimean Congo virus, Toscana virus, ZIKA virus, Chickungunya virus (CHIKV), Akabane virus (AKAV), Schmallenberg virus (SBV), influenza (such as Influenza A H3N2, or H1N1 virus), adeno-associated virus (AAV), Newcastle disease virus (NDV), or vesicular stomatitis virus (VSV). In an embodiment, the surface contaminated by a virus, or suspected of being contaminated by a virus, is found on medical equipment, furniture or clothing, or in medical rooms such as operating theatres or hospital wards.

In another aspect, the invention provides a device for sterilisation or viral disinfection comprising a composition as defined herein and means for dispensing the composition. In an embodiment, the device is a spray and the means comprise a spray applicator. In an alternative embodiment, the means comprise a support material impregnated with the composition. Suitably the support material is a textile, a woven or non-woven fabric, an absorbent sheet or a sponge.

In another aspect, the invention provides a material comprising a branched polymer as defined herein, or a salt or salts thereof. In an embodiment, the material is impregnated or coated with the branched polymer. In an alternative embodiment, the branched polymer is physically incorporated into the structure of the material; for example, the material is fabricated from the branched polymer, or the branched polymer is covalently bonded to the material. The material may be any material in which virustatic or virucidal activity might be desirable. In an embodiment, the material is selected from a dressing (such as a wound dressing, bandage or the like), clothing (such as protective clothing, e.g. surgical wear, hospital gowns or personal protective wear - such as face masks), or a fabric (such as hospital sheets, curtains and the like).

### LIST OF FIGURES

FIG. 1 shows the ¹H-NMR spectrum in D₂O of Example 9.4 (4-arm PSS DP100)

FIG. 2 shows the HSV-2 plaque forming units/ml (log-scale) following incubation with (A) Example 9.1 (4-arm PSS DP10); (B) Example 9.2 (4-arm PSS DP30); (C) Example 9.3 (4-arm PSS DP50); and (D) Example 9.4 (4-arm PSS DP100) at 5 µg and 15 µg versus the untreated control sample (labelled HSV-2).

FIG. 3 shows the HSV-2 percentage infection at increasing concentrations of Example 9.1 (4-arm PSS DP10), Example 9.2 (4-arm PSS DP30), Example 9.3 (4-arm PSS DP50) and Example 9.4 (4-arm PSS DP100).

FIG. 4 shows the SARS-CoV-2 percentage infection at increasing concentrations of Example 9.3 (4-arm PSS DP50).

FIG. 5 shows the SARS-CoV-2 percentage infection at increasing concentrations of Example 9.3 (4-arm PSS DP50) and Example 9.4 (4-arm PSS DP100) in a separate assay to FIG. 4.

FIG. 6 shows the effect of Example 9.4 (4-arm PSS DP100) on the viral titre (expressed as log10 TClD₅₀ titre reduction) for HSV-2 and RSV at different concentrations.

FIG. 7 shows the HSV-2 plaque forming units/ml (log-scale) following incubation with untreated control sample (NTC); comparative example 11.1 (linear DP100 PSS polymer with CTA), comparative example 11.2 (linear DP100 PSS polymer without CTA); Example 9.4 (4-arm PSS DP100); and comparative example 11.3 (cleaved 4-arm PSS DP100).

FIG. 8 shows the results of the MTS cell health assay using 50 to 500 µg/mL concentrations of (left to right): cells only; Example 9.1; Example 9.2; Example 9.3; and Example 9.4

### EXAMPLES

### General Procedures

Methods for preparing the compounds of this invention are illustrated in the following Examples. Starting materials are made according to procedures known in the art or as illustrated herein, or are available commercially. Commercial reagents were used without further purification. Where no reaction temperature is included, the reaction was performed at ambient temperature which is typically 18-27° C.

Where compounds described in the invention are characterized by ¹H NMR spectroscopy, spectra were recorded on 400 MHz instruments. Where no temperature is included the spectra were recorded at ambient temperature. Chemical shift values are expressed in parts per million (ppm).

Abbreviations:

| | |
|---|---|
| ACVA | 4,4′-azobis(4-cyanovaleric acid) |
| AIBN | azobis(isobutyronitrile) |
| ATRP | atom transfer radical polymerisation |
| CTA | chain transfer agent |
| DCM | dichloromethane |
| DP | degree of polymerisation |
| EtOH | ethanol |
| HSV | herpes simplex virus |
| MeOH | methanol |
| MWCO | molecular weight cut off |
| MTS | 3-(4,5-dimethylthiazol-2-yl)-5-(3-carboxymethoxyphenyl)-2-(4-sulfophenyl)-2H-tetrazolium |
| NMR | nuclear magnetic resonance |
| PBS | phosphate buffered saline |
| PFU | plaque forming units |
| PSS | polystyrene-4-sulfonate |
| RAFT | reversible addition fragmentation chain transfer polymerisation |
| RO | reverse osmosis (purified) |
| RSV | respiratory syncytial virus |
| RT | room temperature |
| SARS-CoV | severe acute respiratory syndrome coronavirus |
| SPMA | 3-sulfopropylmethacrylate |

### Synthesis of Branched Polymers

### Example 1: Synthesis of 4-Arm ATRP Initiator

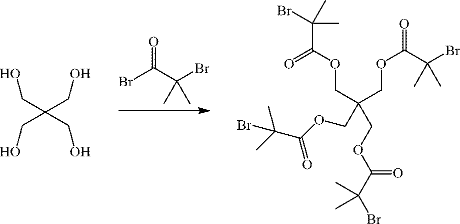

To pentaerythritol (2.4 g, 1 eq) was added 20 mL pyridine and the mixture was cooled to 0° C. 2-bromoisobutyryl bromide (24.3 g, 6 eq) added drop wise followed by stirring at 0° C. for 30 min and the reaction mixture was allowed to warm to room temperature overnight with stirring. After concentration under reduced pressure DCM (100 mL) was added followed by washing against NaHSO₄ (1 M; 3 × 50 mL), Na₂CO₃ (10%; 3 × 50 mL) and brine (3 × 50 mL). The organic layer was then dried over MgSO₄ before drying under reduced pressure. The resultant solid was recrystallised in MeOH to yield the pure product in quantitative yield.

### Example 2: Synthesis of 6-Arm ATRP Initiator

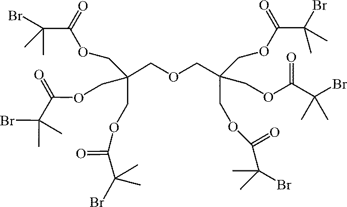

To di-pentaerythritol (2.54 g, 1 eq) was added 20 mL pyridine and the mixture was cooled to 0° C. 2-bromoisobutyryl bromide (20.7 g, 9 eq) added drop wise followed by stirring at 0° C. for 30 min and the reaction mixture was then allowed to warm to room temperature overnight with stirring. After concentration under reduced pressure DCM (100 mL) was added followed by washing against NaHSO₄ (1 M; 3 × 50 mL), Na₂CO₃ (10%; 3 × 50 mL) and brine (3 × 50 mL). The organic layer was then dried over MgSO₄ before drying under reduced pressure. The resultant solid was recrystallised in MeOH to yield the pure product in quantitative yield.

### Example 3: Synthesis of 4-Arm PSS Via ATRP Polymerisation Using 4-Arm ATRP-Initiator

Sodium 4-vinylbenzenesulfonate (4n mol eq*) was combined with 4-arm ATRP-initiator (Example 1) (44.4 mg, 1 eq)in H₂O:EtOH (3:1) and degassed by N₂ bubbling. Separately CuCl (24 mg) and tris[2-(dimethylamino)ethyl]amine (Me₆TREN) (55.86 mg) were combined in H₂O:EtOH (3:1) and degassed by N₂ bubbling. After approximately 30 min the copper mixture was added to the stirred monomer solution (under N₂). The reaction was left for 24 hrs at room temperature before being precipitated into acetone. The resultant solid was dialysed (1000 MWCO) against a basic ethylenediaminetetraacetic (EDTA) solution followed by dialysis against RO H₂O. *wherein n is the degree of polymerisation or the number of PSS residues. Therefore, when n = 50, 200 mol eqv of sodium 4-vinylbenzenesulfonate are used.

### Example 4: Synthesis of 6-Arm PSS Via ATRP Polymerisation Using 6-Arm ATRP-Initiator

Sodium 4-vinylbenzenesulfonate (6n mol eq*) was combined with 6-arm ATRP-initiator (Example 2) (46.4 mg, 1 eq) in H₂O—EtOH (3:1) and degassed by N₂ bubbling. Separately CuCl (24 mg) and tris[2-(dimethylamino)ethyl]amine (Me₆TREN) (55.86 mg) were combined in H₂O—EtOH (3:1) and degassed by N₂ bubbling. After approximately 30 min the copper mixture was added to the stirred monomer solution (under N₂). The reaction was left for 24 hrs at room temperature before being precipitated into acetone. The resultant solid was dialysed (1000 MWCO) against a basic ethylenediaminetetraacetic (EDTA) solution followed by dialysis against RO H₂O. *wherein n is the degree of polymerisation or the number of PSS residues. Therefore, when n = 50, 300 mol eqv of sodium 4-vinylbenzenesulfonate are used.

### Example 5: Synthesis of 4-Arm CTA-OH

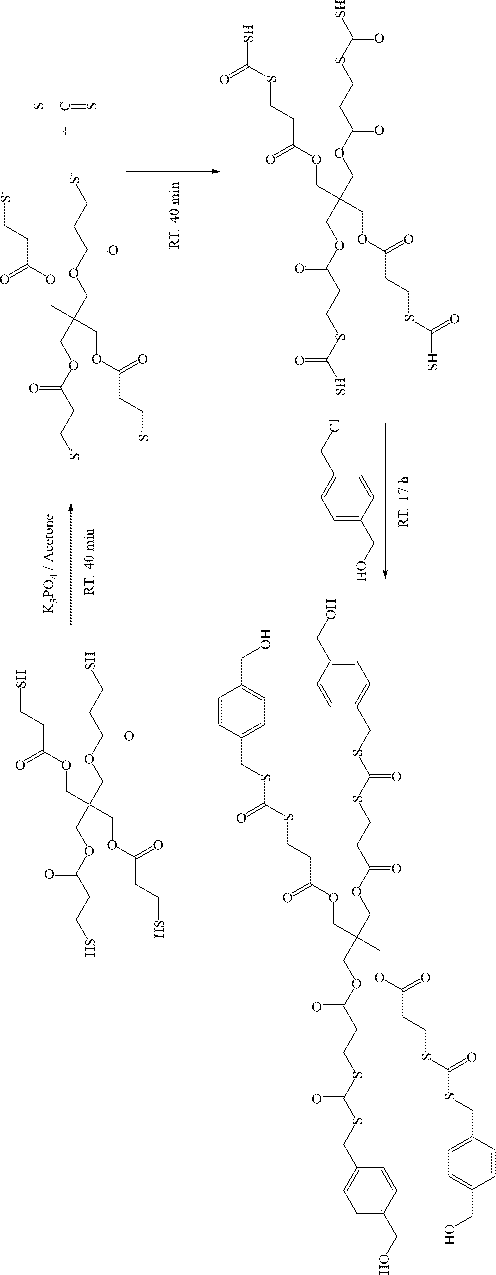 [SMILES: O=C(CCS)OCC(COC(=O)CCS)(COC(=O)CCS)COC(=O)CCS ; CC(SC(=S)SCCC(=O)OCC(COC(=O)CCSC(=S)SC(C)C(=O)O)(COC(=O)CCSC(=S)SC(C)C(=O)O)COC(=O)CCSC(=S)SC(C)C(=O)O)C(=O)O]

Pentaerythritol tetra-(3-mercaptopropionate) (0.50 g, 1.02 mmol, 1 eq) was added to a stirred suspension of potassium phosphate (912 mg, 6.70 mmol, 6.6 eq) in 15 ml of acetone and stirred for 45 min at room temperature. Then, carbon disulphide (0.913 g, 11.99 mmol, 11.8 eq) was added to the previous solution and stirred for 45 min at room temperature. 4-chloromethyl(benzoyl) alcohol (689 mg, 4.40 mmol, 4.3 eq) was added and stirred at RT overnight. The reaction mixture was filtered and washed with acetone. Sample was then dried under reduced pressure. The solid was then dissolved in DCM, washed with water and brine several times and then the organic phase dried over magnesium sulphate. Column chromatography in DCM gave the pure product.

### Example 6: Synthesis of 6-Arm CTA-OH

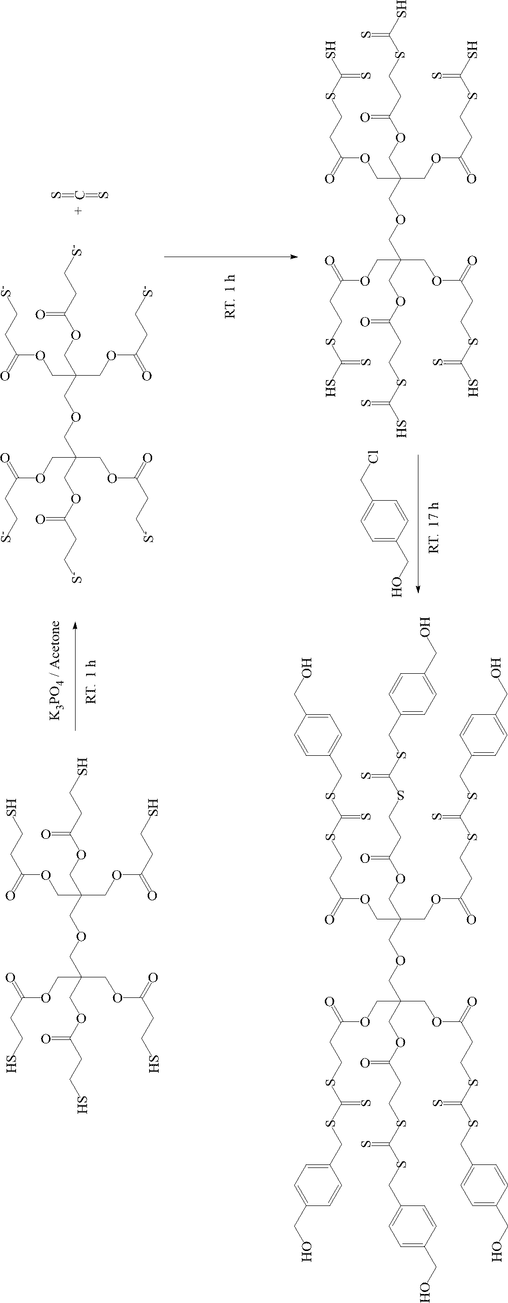

Dipentaerythritol hexakis-(3-mercaptopropionate) (1.00 g, 1.28 mmol, 1 eq) was added to a stirred suspension of potassium phosphate (1.716 g, 12.61 mmol, 9.9 eq) in 20 ml of acetone and stirred for 45 min at room temperature. Then, carbon disulphide (1.748 g, 22.95 mmol, 17.9 eq) was added to the previous solution and stirred for 45 min at room temperature. 4-chloromethyl(benzoyl) alcohol (1.199 g, 7.66 mmol, 6.0 eq) was added and stirred at RT overnight. The reaction mixture was filtered and washed with acetone. Sample was then dried under reduced pressure. The solid was then dissolved in DCM, washed with water and brine several times and then the organic phase dried over magnesium sulphate. Column chromatography in DCM gave the pure product.

### Example 7: Synthesis of 4-Arm CTA-COOH

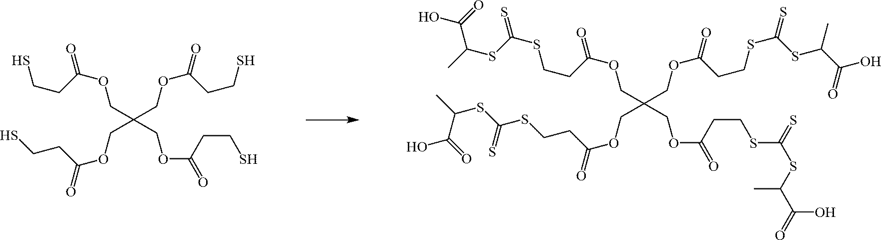

Potassium hydroxide (505 mg, 4.8 eq) was suspended with vigorous stirring in acetone (30 mL) and water (30 mL), followed by the addition of Pentaerythritol tetra-(3-mercaptopropionate) (1.0 g, 2.04 mmol, 1 eq). This mixture was allowed to stir at room temperature for 1 hour before the addition of carbon disulphide (1.59 g, 4.8 eq). After 1 hr of further stirring at room temperature 2-bromopropionic acid (1.38 g, 4.8 eq) was added, after which the reaction was allowed to stir overnight at room temperature. The reaction was stopped and filtered, the remaining solution was evaporated under reduced pressure, before being re-dissolved in dichloromethane (150 ml) and washed repeatedly against deionised water (3 × 200 ml), the aqueous layers were combined and concentrated under reduced pressure. The concentrated solution was then dialysed against several batches of deionised water, before being evaporated under reduced pressure and finally freeze-dried to recover a solid material. The resultant solid was then acidified with acetic acid before being purified by column chromatography using ethyl acetate:hexane (3:1) with 3% acetic acid as co-eluent.

### Example 8: Synthesis of 6-Arm CTA-COOH

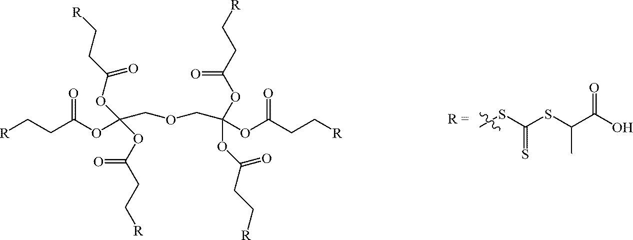

Potassium hydroxide (487 mg, 7.2 eq) was suspended with vigorous stirring in acetone (30 mL) and water (30 mL), followed by the addition of dipentaerythritol hexakis-(3-mercaptopropionate) (1.0 g, 1.28 mmol, 1 eq). This mixture was allowed to stir at room temperature for 1 hour before the addition of carbon disulphide (1.53 g, 7.2 eq). After 1 hr of further stirring at room temperature 2-bromopropionic acid (1.33 g, 7.2 eq) was added, after which the reaction was allowed to stir overnight at room temperature. The reaction was stopped and filtered, the remaining solution was evaporated under reduced pressure, before being re-dissolved in dichloromethane (150 ml) and washed repeatedly against deionised water (3 × 200 ml), the aqueous layers were combined and concentrated under reduced pressure. The concentrated solution was then dialysed against several batches of deionised water, before being evaporated under reduced pressure and finally freeze-dried to recover a solid material. The resultant solid was then acidified with acetic acid before being purified by column chromatography using ethyl acetate:hexane (3:1) with 3% acetic acid as co-eluent.

### Example 9: Synthesis of 4-Arm PSS via RAFT Polymerisation Using 4-Arm CTA-COOH

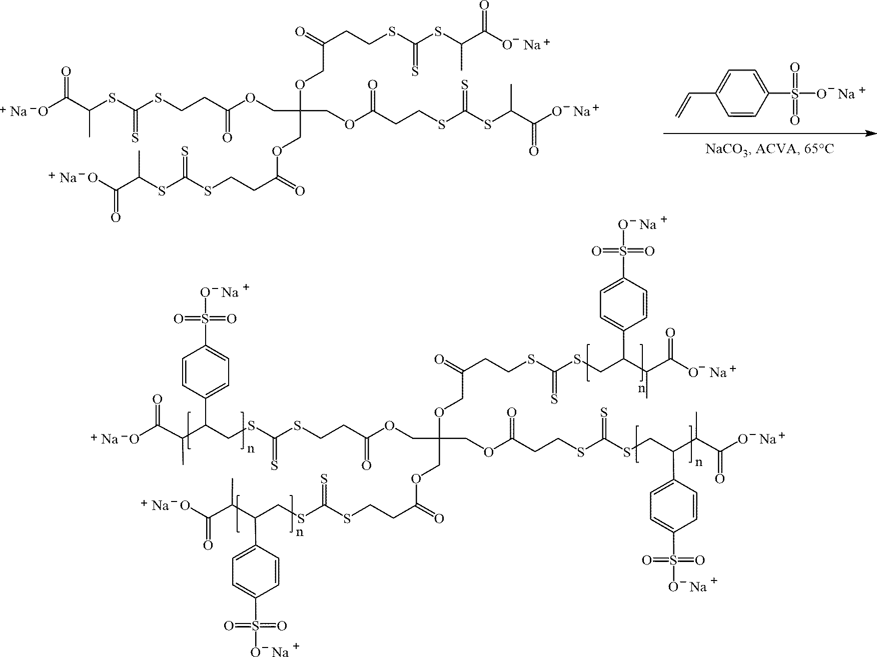

4-arm CTA-COOH (43.7 mg, 1 mol eq), 0.5 M sodium carbonate solution, sodium 4-vinylbenzenesulfonate (4n mol eq*) and 4,4′-azobis(4-cyanopentanoic acid) (ACVA) (1.13 mg) were combined in H₂O (4 ml) and sealed and degassed. The reaction mixture was then stirred at 70° C. for 4 hr. After complete polymerisation the reaction mixture was rapidly cooled and the polymer precipitated into acetone, before being dialysed (1000 MWCO) against H₂O and dried to yield pure polymer. *For example, for the 4-arm PSS DP10, 10 mol eqv of sodium 4-vinylbenzenesulfonate were used for each arm, therefore 40 mol eqv were used relative to the number of moles of CTA.

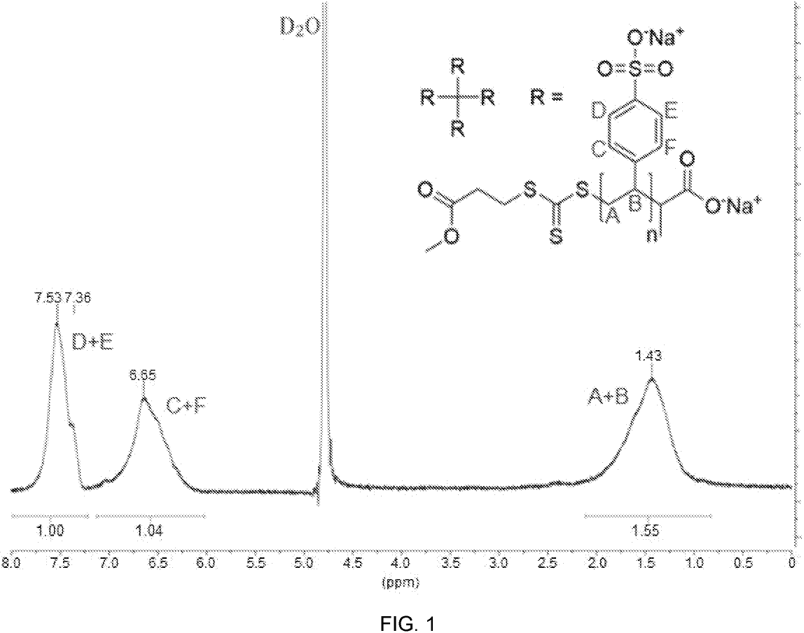

The branched polymers prepared according to Example 9 are summarised in Table A below. FIG. 1 shows a ¹H-NMR spectrum of Example 9.4.

### Example 10: Synthesis of 4-Arm SPMA via RAFT Polymerisation Using 4-Arm CTA-COOH

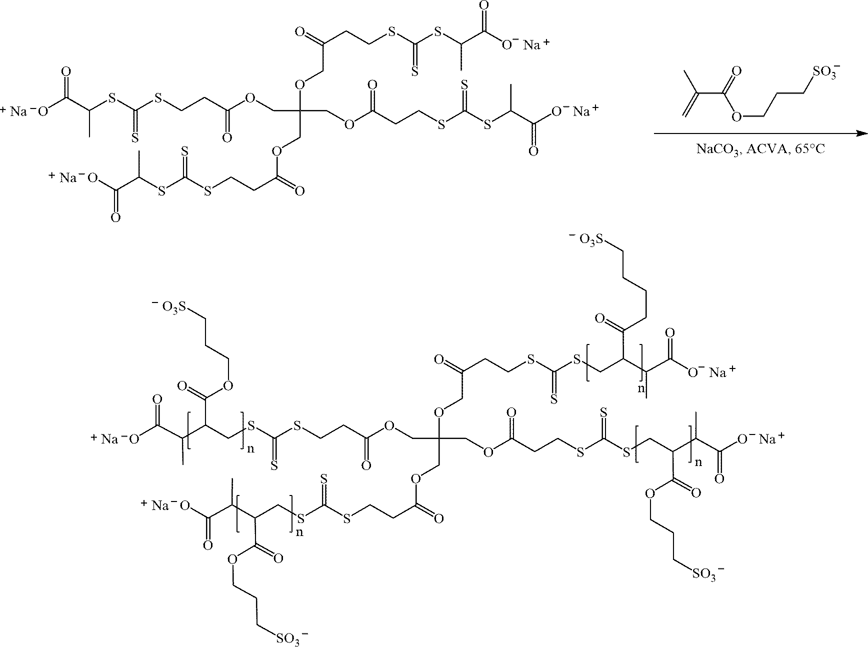

4-arm CTA-COOH (36.6 mg, 1 mol eq), 3-sulfopropyl methacrylate (4n mol eq*) and 4,4′-azobis(4-cyanopentanoic acid) (ACVA) (0.95 mg) were combined in H₂O (4 ml) and sealed and degassed. The reaction mixture was then stirred at 70° C. for 4 hr. After complete polymerisation the reaction mixture was rapidly cooled and the polymer precipitated into acetone, before being dialysed (1000 MWCO) against H₂O and dried to yield pure polymer. *For example, for the 4-arm SPMA DP30, 30 mol eqv of 3-sulfopropyl methacrylate were used for each arm, therefore 120 mol eqv were used relative to the number of moles of CTA.

The branched polymers prepared according to Example 10 are summarised in Table A below.

TABLE A

| Ex. No. | Description | 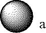 | X | n | q | Z^{d} | m |
|---|---|---|---|---|---|---|---|
| 9.1 | 4-arm PSS DP10 | A | PSS^{b} | 10 | 0 | Z1 | 4 |
| 9.2 | 4-arm PSS DP30 | A | PSS^{b} | 30 | 0 | Z1 | 4 |
| 9.3 | 4-arm PSS DP50 | A | PSS^{b} | 50 | 0 | Z1 | 4 |
| 9.4 | 4-arm PSS DP100 | A | PSS^{b} | 100 | 0 | Z1 | 4 |
| 10.1 | 4-arm SPMA DP10 | A | SPMA^{c} | 10 | 0 | Z1 | 4 |
| 10.2 | 4-arm SPMA DP30 | A | SPMA^{c} | 30 | 0 | Z1 | 4 |
| 10.3 | 4-arm SPMA DP50 | A | SPMA^{c} | 50 | 0 | Z1 | 4 |
| 10.4 | 4-arm SPMA DP100 | A | SPMA^{c} | 100 | 0 | Z1 | 4 |

^{a} Cores: 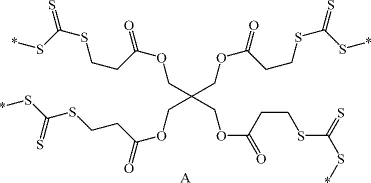
^{b} PSS = polystyrene-4-sulfonate, 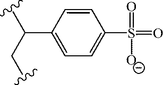
^{c} SPMA = 3-sulfopropylmethacrylate, 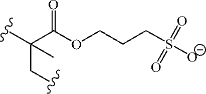
^{d} Capping Groups:

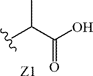

### Comparative Example 11: Linear Polymers and Branched Cleaved Polymer

To evaluate the antiviral activity of comparative non-branched polymers, the following materials were prepared:

### Comparative Example 11.1 - Linear DP100 (n = 100) PSS Polymer Incorporating Chain Transfer Agent

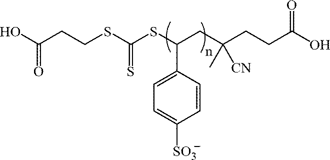

### Comparative Example 11.2 - Linear DP100 (n = 100) PSS Polymer Without Chain Transfer Agent

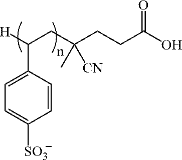

### Comparative Example 11.3 - Branched Cleaved DP100 (n = 100) PSS Polymer

Example 9.4 branched polymer (4-arm PSS DP100), prepared as described hereinabove, was subjected to chemical cleavage of the trithiocarbonate moieties to yield a derivative wherein the sulfonated residues have been cleaved from the 4-arm core structure. This was achieved by heating Example 9.4 for 3 hours at 75° C. in the presence of N-ethylpiperidine hypophosphite and AIBN in water. After cooling, the cleaved material was dialysed for 3 days with twice daily changes of water.

### Biological Assays

### Inhibition Assay With HSV-2

The effect of antiviral branched polymers on HSV-2 infection was evaluated by a plaque reduction assay. Vero cells were seeded 24 hours in advance in 24-well plates at a density of 10⁵ cells per well. Increasing concentrations of antivirals were incubated with HSV-2 [multiplicity of infection (MOI), 0.0003 plaque-forming units (PFU)/cell] at 37° C. for 1 hour, and then the mixtures were added to the cells. Following virus adsorption (2 hours at 37° C.), the virus inoculum was removed and the cells were washed with medium and then overlaid with a medium containing 1.2% methylcellulose. After incubation with HSV-2 for 24 hours, respectively, at 37° C., cells were fixed and stained with 0.1% of crystal violet in 20% ethanol and viral plaques were counted. The concentration of compound producing 50% reduction in plaque formation (EC₅₀) was determined using Prism software by comparing drug-treated and untreated wells.

### Evaluation of Virucidal Activity Against HSV-2 and RSV-A

Viruses (10⁵ PFU for HSV-2) and antivirals (15 µg/ml) were incubated for 1 hour at 37° C., and then the virucidal effect were investigated with serial dilutions of the mixtures over a 96 well plate seeded with Vero cells (95% confluent). Following incubation, virus/antiviral mixtures were diluted 1:10, 1:100 and 1:1000 and then 50 µL from each (in duplicate) was added per well (already containing 100 µL of 2% FBS media), before being serially diluted down the plate. Viral titers were calculated at dilutions at which the antiviral was not effective.

### Inhibition Assay With SARS-CoV-2 (A)

The effect of the branched polymers Example 9.3 and Example 9.4 on SARS-CoV-2 infection was evaluated by a plaque reduction neutralisation assay.

Test compound (Example 9.3 or Example 9.4) was serially diluted, in duplicate and each dilution was incubated with approximately 40 PFU of wild type SARS-CoV-2 (2019-nCoV/Victoria/1/2020) virus, for 1 h at 37° C. The samples were then allowed to absorb for 1 hour at 37° C. on Vero E6 [Vero 76, clone E6 (ECACC 85020206), European Collection of Authenticated Cell Cultures, UK] monolayers in 24-well plates. Afterwards plaque assay overlay media was added and the samples were incubated for at 37° C. for 5 days. Then plates were fixed overnight with 20% (w/v) formalin/PBS, washed with tap water and stained with methyl crystal violet solution (0.2% v/v) and the viral plaques were counted.

An internal negative control using serial dilutions of test compound with Vero E6 cells only, was run in parallel for 5 days to monitor cell monolayer integrity/toxicity caused by test compound. An internal positive control, was run in parallel, using a sample of human MERS convalescent serum known to neutralise SARS-CoV-2 (National Institute for Biological Standards and Control, UK).

### Inhibition Assay With SARS-CoV-2 (B)

The effect of the branched polymers Example 9.3 and Example 9.4 on SARS-CoV-2 infection was evaluated by an alternative plaque reduction assay.

The media was aspirated from the cells (max 6 wells per time, to avoid drying) and 200 µl of a mixture of SARS-CoV-2 virus and test compound (Example 9.3 or Example 9.4) was added per well (each dilution in duplicate). The 24-well plate was incubated for 1 h at 37° C.

The solution was aspirated (max 6 wells per time, to avoid drying) and 500 µl of Avicel-rich DMEM solution (containing 0.33% of Avicel3515 and 5%FBS) was added. The plates were covered with a film and incubated at 37° C. for 48 h.

The solution was aspirated (max 6 wells per time, to avoid drying) and 500 µl of formalin (4% PFA) was added. After 15-30 minutes the solution was aspirated (max 6 wells per time, to avoid drying) and 500 µl of Crystal Violet solution was added. After 15-30 minutes the solution was aspirated and 500 µl of water was added to wash.

The solution was aspirated again and then incubated at 70° C. for 30-60 min. The viral plaques were then counted.

### TCID₅₀ Viral (HSV-2/RSV) Titre Reduction Assay Using Example 9.4

A flask of Vero cells was split with trypsin for seeding 96 well plates. Plates were seeded with Dulbecco’s modified eagle’s media (DMEM) and cells at a density of 5×10⁵/ml. All wells of the plate contained 100 µl of cell and media mixture, before an additional 80 µl of cell and media mixture was added to the first four wells of the left-most column.

Once seeded, plates were left for ninety minutes for cells to adhere. One plate in each experimental batch was utilised as a positive control (virus + sterile deionised water). Subsequently, using sterile Eppendorf tubes, 55 µl of virus stock solution was mixed together with 55 µl of Example 9.4 solution (or water) for five minutes contact time at room temperature. Mixtures were pipetted up and down to ensure virus stock and test solution were thoroughly mixed. 20 µl of the mixture was then added to the first four wells of the first column on the appropriate plate (bringing the total volume within the well to 200 µl).

These four wells were then titrated across the plates using a 1-in-2 serial dilution (100 µl from the first set of four wells into the second set, 100 µl from the second set into the third set, continuing across the plate but leaving the last column untouched as a negative control). Dilutions from the top four wells of the penultimate column were transferred to the bottom four wells of the first column and serial dilution continued.

Finally, cytopathic effects were scored after an appropriate length of time for the relevant virus (7 days for RSV, 2 days for HSV-2) and titres calculated from these observations using the Spearman-Karber method for determining endpoint dilution.

### MTS Assay for Cell Health Using Examples 9.1-9.4

HeLa cells were cultured in a 96-well microtiter plate until confluent (16,000 cells per well). Media was discarded and 50µl of fresh minimum essential medium Eagle (MEME) was added. 50 µl of test material (Example 9.1, 9,2, 9.3 or 9.4) was added to give 100 µl final volume in each well with a concentration range from 50 µg/mL to 500 µg/mL.

The plate was incubated for 24 h and then 20 µl of CellTiter 96® AQueous One Solution Reagent was pipetted into each well. The plate was incubated at 37° C. for 4 hours in a humidified, 5% CO2 atmosphere and then the absorbance at 490 nm was recorded using a 96-well plate reader.

### Biological Assay Results

### HSV-2 Assay Results

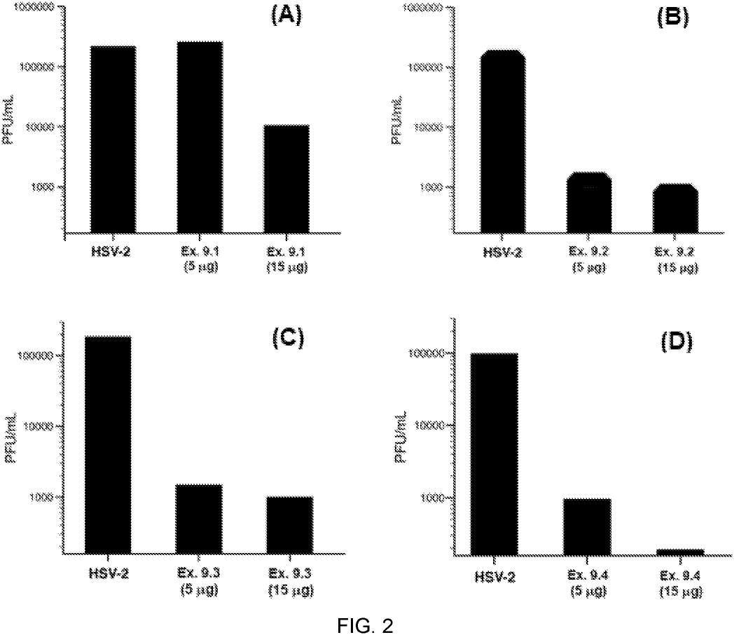

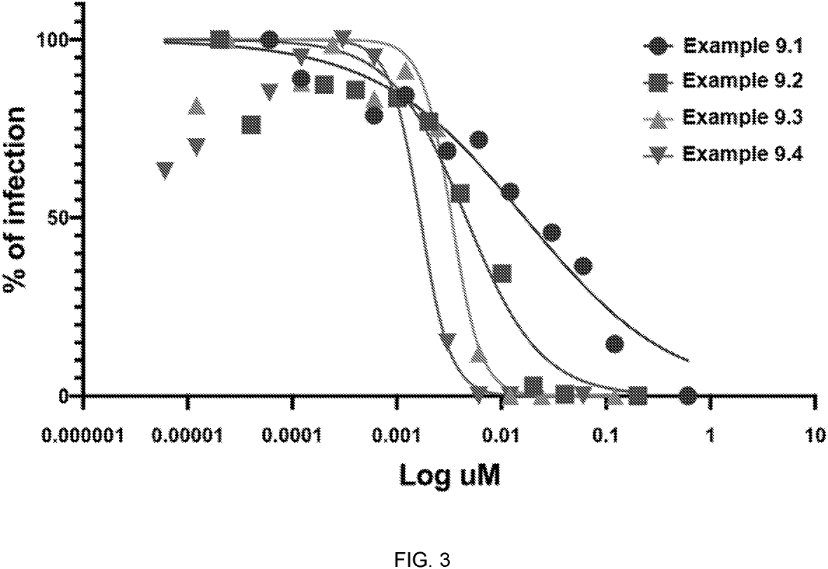

The antiviral activity of the branched polymers tested against HSV-2 can be seen in Table B and FIGS. 2 and 3.

TABLE B

| Example Number | Description | HSV-2 IC₅₀ (ng/ml) | HSV-2 IC₉₀ (ng/ml) |
|---|---|---|---|
| 9.1 | 4-arm PSS DP10 | 138.1 | 4899 |
| 9.2 | 4-arm PSS DP30 | 113.6 | 690.9 |
| 9.3 | 4-arm PSS DP50 | 137.4 | 281.2 |
| 9.4 | 4-arm PSS DP100 | 138.1 | 288.0 |

It can be seen that all the 4-arm PSS polymers tested demonstrated antiviral activity against HSV-2. FIG. 2 shows that Examples 9.2 (4-arm PSS DP30), 9.3 (4-arm PSS DP50) and 9.4 (4-arm PSS DP100) gave greater than 2 log units decrease in HSV-2 PFU/mL at both 5 µg and 15 µg incubation. A greater than 2 log units decrease in viral PFU/mL compared to control is typically seen as being indicative that an agent has virucidal activity. Therefore, the DP30-100 PSS branched polymers exhibit virucidal activity against HSV-2.

From FIG. 3 and Table B it can be seen that all of the 4-arm PSS polymers exhibited similar IC₅₀ values against HSV-2 in the range 114-138 ng/ml. However, 90% inhibition (IC₉₀) correlated with the number of PSS residues, whereby the polymers having longer blocks of sulfonated residues (Example 9.3 - DP50 and Example 9.4 -DP100) gave more effective inhibition (IC₉₀ 280-290 ng/ml) than the polymers having shorter blocks of sulfonated residues, namely Example 9.1 - DP10 (IC₉₀ 4900 ng/ml) and Example 9.2 - DP30 (IC₉₀ 691 ng/ml).

The 4-arm SPMA polymers (Examples 10.1 to 10.4) were tested in the same HSV-2 assay and were all found to have at least virustatic activity.

### SARS-CoV-2 Assay Results

The antiviral acivity of the 4-arm PSS DP50 (Example 9.3) branched polymer was tested against SARS-CoV-2 using the inhibition assay (A) protocol, as can be seen in Table C.

TABLE C

Viral plaque count

| Sample | 3 µg/ml | 6 µg/ml | 12.5 µg/ml | 25 µg/ml | 50 µg/ml | 100 µg/ml |
|---|---|---|---|---|---|---|
| Ex. 9.3 | 0 | 0 | 0 | 0 | 0 | 0 |
| Ex. 9.3 | 0 | 0 | 0 | 0 | 0 | 0 |
| Ex 9.3 + Virus | 13 | 9 | 13 | 14 | 13 | 4 |
| Ex 9.3 + Virus | 10 | 9 | 8 | 10 | 8 | 2 |
| Virus | - | 34 | 35 | 37 | 31 | 36 |

The vero cell monolayers in the test compound-only negative control samples were healthy and no viral plaques were observed as expected. The number of plaques in the samples containing both test compound and virus were lower than the virus-only control wells, indicating that the test compound (Example 9.3) has antiviral activity against SARS-CoV-2.

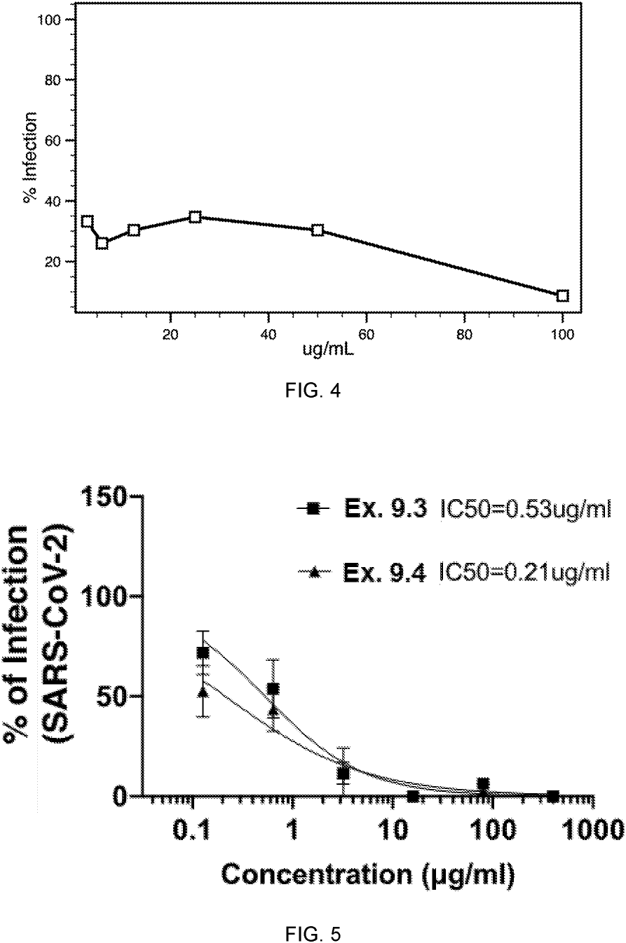

From the data in Table C it can be seen that compared to an average viral plaque count from the virus-only control samples of 34.6, for the samples containing both test compound and virus, the average % inhibition from duplicate runs was 33% at 3 µg/ml test compound, 26% at 6 µg/ml, 30% at 12.5 µg/ml, 35% at 25 µg/ml, 30% at 50 µg/ml and 9% at 100 µg/ml. This % inhibition data against SARS-CoV-2 is shown in FIG. 4.

The antiviral acivity of the 4-arm PSS DP50 (Example 9.3) and 4-arm PSS DP100 (Example 9.4) branched polymers was also tested against SARS-CoV-2 using the inhibition assay (B) protocol; the results can be seen in FIG. 5. Both branched polymers showed good inhibition of the SARS-CoV-2 virus with the DP100 polymer giving the greatest inhibition (IC₅₀ = 0.21 µg/mL).

### HSV-2/RSV Titre Reduction Assay Results

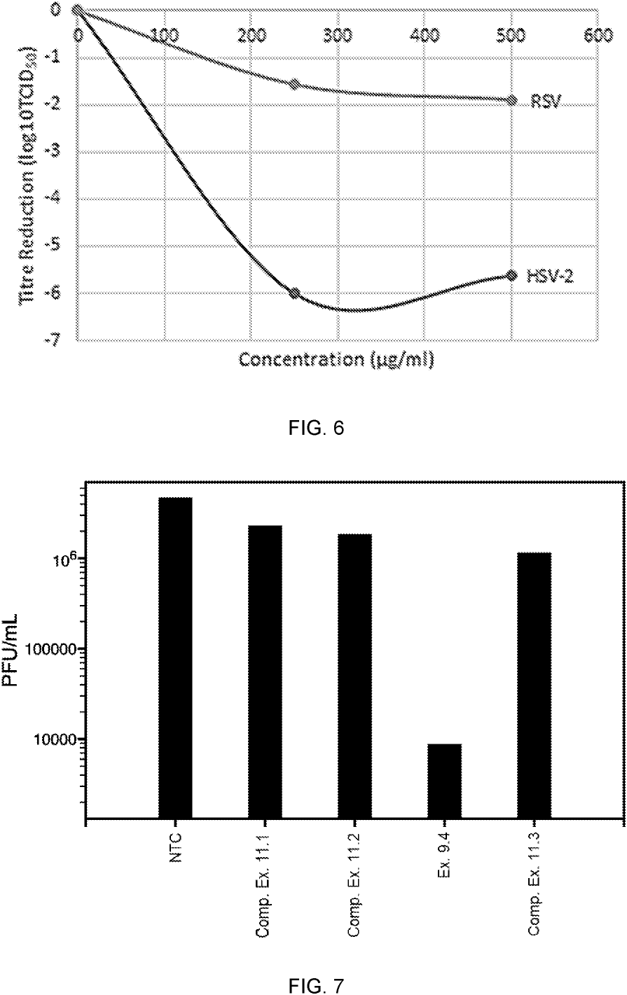

The antiviral activity of the 4-arm PSS DP100 (Example 9.4) branched polymer was tested against HSV-2 and RSV and the results can be seen in FIG. 6. The polymer exhibited virucidal activity against both viruses, with a 2 log unit reduction in viral titre being observed for HSV-2 at less than 100 µg/mL, and for RSV at approximately 500 µg/mL.

### HSV-2 PFU Assay - Effect of Branched Versus Non-branched PSS Polymers

FIG. 7 shows that only the branched sulfonated polymer according to the present invention (Example 9.4 - DP 100) possessed virucidal activity (at least 2 log unit reduction in viral PFU). When linear sulfonated polymers having DP 100 were tested in the same assay they showed only virustatic activity. The presence (comparative example 11.1) or absence (comparative example 11.2) of the chain transfer agent in the structure of the linear polymer was shown to have no significant effect on the antiviral activity.

To further corroborate this finding, after Example 9.4 had been subjected to cleavage of the branches (comparative example 11.3), the virucidal activity was lost.

This data indicates that in order to provide sulfonated polymers having virucidal activity, a branched polymer having a core surrounded by a high density of anionic moieties (sulfonate or sulfate) is needed.

### Cell Health Assay Results

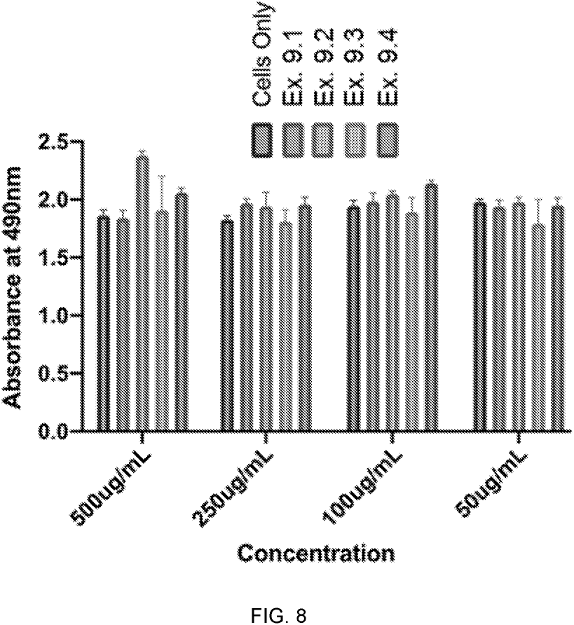

FIG. 8 demonstrates that compared to the control (cells only) none of the branched polymers tested (Examples 9.1 to 9.4) showed any cytotoxic activity, indicating their potential usefulness for clinical applications.

1. A branched polymer according to Formula I: 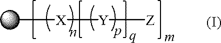
wherein 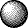
is a polyvalent core structure;
X is a monomer residue comprising at least one sulfonate or sulfate substituent;
Y is a monomer residue;
Z is a capping group;
m is greater than or equal to 3;
n is 5 to 500;
p is 1 to 500; and
q is 0 to 200; wherein if q is greater than 1, then at each occurrence, Y may be the same or different residues.

2. A branched polymer according to claim 1, wherein X has a structure according to Formula II: 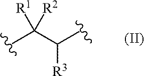
wherein
R¹ is aryl, C₁₋₂₀alkyl, C₁₋₂₀alkylene-aryl, C(O)OR⁴, C(O)NHR⁴, OC(O)R⁴, NHC(O)R⁴, or sulfonate;
R² and R³ are independently selected from hydrogen and C₁₋₄alkyl; and
R⁴ is C₁₋₁₀alkyl or aryl; and wherein
each of the aryl, C₁₋₂₀alkyl, C₁₋₂₀alkylene-aryl, or R⁴ groups is substituted with one or more sulfonate or sulfate groups, and is optionally substituted with one or more substituents selected from hydroxy, halo, C₁₋₄alkyl, C₁₋₄alkoxy, aryl, and cyano.

3. A branched polymer according to claim 2, wherein R¹ is phenyl, C(O)OR⁴, or C(O)NHR⁴, wherein R⁴ is C₁₋₁₀alkyl and each of the phenyl or R⁴ groups is substituted with one or more sulfonate groups and is optionally substituted with one or more substituents selected from hydroxy, halo, C₁₋₄alkyl, C₁₋₄alkoxy, and cyano.

4. A branched polymer according to claim 2, wherein R¹ is selected from: 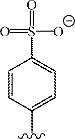 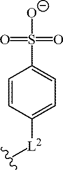 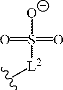 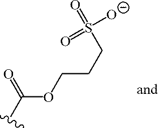 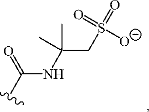
wherein L
² is C₁₋₂₀alkylene.

5. A branched polymer according to any one of claims 2 to 4, wherein R² and R³ are independently selected from hydrogen and methyl,.

6. A branched polymer according to any one of claims 2 to 4, wherein R² is hydrogen or methyl and R³ is hydrogen.

7. A branched polymer according to any one of claims 1 to 6, wherein n is 5 to 400, such as 5 to 300, 10 to 250, or conveniently 20 to 100.

8. A branched polymer according to any one of claims 1 to 7, wherein Z is selected from hydrogen, hydroxyl, bromo, chloro, or one of the following groups: 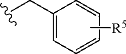 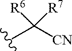 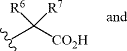 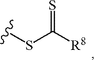
wherein:
R⁵ is hydrogen, halo, cyano, CO₂H, C₁₋₃haloalkyl, C₁₋₃alkylene-OH, or C₁₋₃alkylene-NH₂;
R⁶ and R⁷ are independently chosen from hydrogen and C₁₋₃alkyl optionally substituted with cyano, halo, or CO₂H;
R³ is S-C₁₋₁₅alkyl, S-aryl, NR⁹R¹⁰ or aryl, said C₁₋₁₅alkyl and aryl groups being optionally substituted with one or more substituents selected from hydroxy, C₁₋₃alkyl, halo, cyano, CO₂H, and C₁₋₃haloalkyl;
R⁹ is hydrogen or C₁₋₃alkyl; and
R¹⁰ is hydrogen, C₁₋₁₂alkyl or aryl, said C₁₋₁₂alkyl and aryl groups being optionally substituted with one or more substituents selected from hydroxy, C₁₋₃alkyl, halo, cyano, CO₂H, and C₁₋₃haloalkyl.

9. A branched polymer according to any one of claims 1 to 7, wherein Z is selected from one of the following groups: 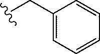 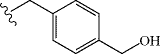 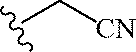 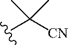 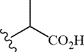 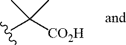 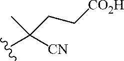
.

10. A branched polymer according to any one of claims 1 to 9, wherein p is 5 to 400, such as 5 to 300, 10 to 250, or conveniently 20 to 150.

11. A branched polymer according to any one of claims 1 to 10, wherein q is 0, or q is 1.

12. A branched polymer according to any one of claims 1 to 11, wherein m is 3 to 12, such as 4 to 10, or conveniently 4 or 6.

13. A branched polymer according to any one of claims 1 to 11, wherein 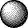
is selected from: 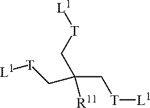 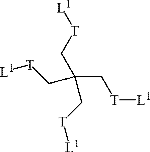 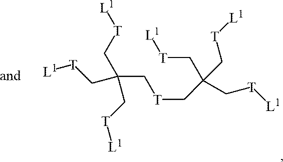
wherein
T is O, S or NH;
R¹¹ is hydrogen or C₁₋₄alkyl; and
L¹ is selected from one of the following linker groups: 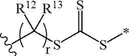 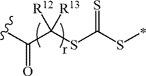 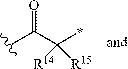 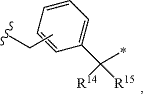
and wherein 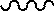
is the point of attachment to T and * is the point of attachment to an X residue;
R¹² and R¹³ are independently chosen from hydrogen, C₁₋₃alkyl, hydroxy, and halo;
r is 1 to 10; and
R¹⁴ and R¹⁵ are independently chosen from hydrogen and C₁₋₃alkyl.

14. A branched polymer according to claim 13, wherein L¹ is selected from: 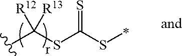 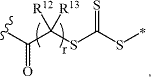
and Z is selected from: 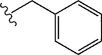 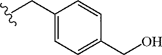 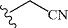 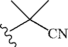 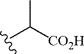 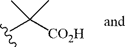 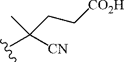
.

15. A branched polymer according to claim 13, wherein L¹ is 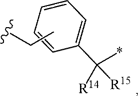
and Z is: 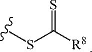
.

16. A branched polymer according to claim 13, wherein L¹ is 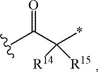
and Z is bromo or chloro.

17. A branched polymer according to any one of claims 13 to 16, wherein T is O.

18. A branched polymer according to claim 1 wherein X is: 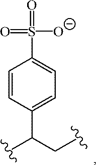
n is 10 to 100, q is 0, and m is 4.

19. A composition comprising a branched polymer according to any one of claims 1 to 18, or a salt or salts thereof.

20. A method of sterilisation or viral disinfection, comprising using an effective amount of the composition of claim 19.

21. A device for sterilisation or viral disinfection comprising the composition of claim 19 and means for dispensing the composition.

22. A pharmaceutical composition comprising a branched polymer according to any one of claims 1 to 18, or a pharmaceutically acceptable salt or salts thereof, and one or more pharmaceutically acceptable excipients.

23. A branched polymer according to any one of claims 1 to 18, or a pharmaceutical composition according to claim 22, or a pharmaceutically acceptable salt or salts thereof, for use in the prevention or treatment of viral infections.

24. The branched polymer or pharmaceutical composition for use in the prevention or treatment of viral infections according to claim 23, wherein the viral infection is associated with herpes simplex virus (HSV), adenovirus, adeno-associated virus, human papillomavirus (HPV), respiratory syncytial virus (RSV), dengue virus, norovirus, lentivirus, human immunodeficiency virus (HIV), human cytomegalovirus (HCMV), human metapneumovirus (HMPV), human parainfluenza virus type 3 (HPIV-3), coronavirus (such as MERS-CoV, SARS-CoV, or SARS-CoV-2), foot-and-mouth disease virus, hepatitis B virus, hepatitis C virus, Ebola virus, nipah virus, Rift Valley fever virus, West Nile virus, Crimean Congo virus, Toscana virus, ZIKA virus, Chickungunya virus (CHIKV), Akabane virus (AKAV) or Schmallenberg virus (SBV), influenza (such as Influenza A H3N2 or H1N1 virus), adeno-associated virus (AAV), Newcastle disease virus (NDV), or vesicular stomatitis virus (VSV).

25. A material comprising a branched polymer according to any one of claims 1 to 18, or a salt, or salts, thereof.
